(19) United States
(12) Patent Application Publication
(10) Pub. No.: US 2025/0149190 A1
(43) Pub. Date: May 8, 2025

(54) AI-BASED HOSPITAL ROOM MANAGEMENT

(71) Applicant: Hatchmed Corporation, Seattle, WA (US)

(72) Inventors: Kyrylo Keydanskyy, Seattle, WA (US); Brian Hatch, Seattle, WA (US); Carl Cox, Kailua, HI (US); Christopher N. Andrews, Cape Coral, FL (US)

(73) Assignee: Hatchmed Corporation, Seattle, WA (US)

(21) Appl. No.: 19/015,518

(22) Filed: Jan. 9, 2025

Related U.S. Application Data
(63) Continuation-in-part of application No. 18/334,635, filed on Jun. 14, 2023.
Application No. 18/334,635 is a continuation-in-part of application No. 18/179,113, filed on Mar. 6, 2023.
(60) Provisional application No. 63/317,455, filed on Mar. 7, 2022.

(51) Int. Cl. G16H 80/00; G16H 10/60; G16H 40/20

(52) U.S. Cl. CPC G16H 80/00; G16H 10/60; G16H 40/20

(57) ABSTRACT

A method implemented by a computer system hosting an artificial intelligence (AI) model. The method including receiving, from a first device providing a first type of healthcare related service, a first input related to the first type of healthcare related service. The method further includes receiving, from a second device providing a second type of healthcare related service, a second input related to the second type of healthcare related service. The method further includes generating, by at least using the first input and the second input as part of an input to the AI model, an instruction for a third device providing a third type of healthcare related service, the instruction indicated by an output of the AI model. The method further includes causing, by at least sending the instruction to the third device, the third device to perform the third type of healthcare related service.

### CROSS-REFERENCES TO RELATED APPLICATIONS

This application is a continuation-in-part of U.S. patent application Ser. No. 18/334,635, filed Jun. 14, 2023, which is a continuation-in-part of U.S. patent application Ser. No. 18/179,113, filed Mar. 6, 2023, which claims priority to U.S. Provisional Patent Application No. 63/317,455, filed Mar. 7, 2022, the entire contents of such applications being incorporated by reference for all purposes in their entirety.

### BRIEF DESCRIPTION OF THE DRAWINGS

Various embodiments in accordance with the present disclosure will be described with reference to the drawings, in which:

FIG. 1 illustrates a block diagram of a system according to an embodiment, in accordance with the present disclosure.

FIG. 2 illustrates another block diagram of a system according to an embodiment, in accordance with the present disclosure.

FIG. 3 illustrates another block diagram of a system according to an embodiment, in accordance with the present disclosure.

FIG. 4 illustrates another block diagram of a system according to an embodiment, in accordance with the present disclosure.

FIG. 5 illustrates another block diagram of a system according to an embodiment, in accordance with the present disclosure.

FIG. 6 shows a flow diagram illustrating methods of sending a request to and/or from a server, in accordance with the present disclosure.

FIG. 7 shows a flow diagram illustrating methods of sending a request to and/or from a hub, in accordance with the present disclosure.

FIG. 8 illustrates another exemplary block diagram of a system according to an embodiment, in accordance with the present disclosure.

FIG. 9 illustrates another exemplary block diagram of a system according to an embodiment, in accordance with the present disclosure.

FIG. 10 illustrates another exemplary block diagram of a system according to an embodiment, in accordance with the present disclosure.

FIG. 11 illustrates another exemplary block diagram of a system according to an embodiment, in accordance with the present disclosure.

FIG. 12 illustrates another exemplary block diagram of a system according to an embodiment, in accordance with the present disclosure.

FIG. 13 illustrates another exemplary block diagram of a system according to an embodiment, in accordance with the present disclosure.

FIG. 14 illustrates another exemplary block diagram of a system according to an embodiment, in accordance with the present disclosure.

FIG. 15 illustrates another exemplary block diagram of a system according to an embodiment, in accordance with the present disclosure.

FIG. 16 illustrates another exemplary block diagram of a system according to an embodiment, in accordance with the present disclosure.

FIG. 17 illustrates another exemplary block diagram of a system according to an embodiment, in accordance with the present disclosure.

FIG. 18 shows a flow diagram illustrating methods of authenticating a user and/or device for communication with a hub, server, and/or third party system, in accordance with the present disclosure.

FIG. 19 illustrates another exemplary block diagram of a system according to an embodiment, in accordance with the present disclosure.

FIG. 20 shows a flow diagram illustrating a method of using an Artificial Intelligence (AI) model to receive inputs from devices to generate an instruction for another device, in accordance with the present disclosure.

FIG. 21 illustrates an exemplary block diagram of a system for training AI models according to an embodiment, in accordance with the present disclosure.

### DETAILED DESCRIPTION

The present application relates to server-based healthcare patient services. A healthcare patient service can include providing a service to a patient, where the service is provided at a location of a healthcare facility and can be requested by the patient or by another operator of a device. In the interest of clarity of explanation, the embodiments are described in connection with a nurse call system of a hospital (e.g., for nurse call assistance requests and/or room controls). However, the embodiments are not limited as such and equivalently apply to other types of healthcare patient services and/or other types of healthcare facilities.

Techniques are described that enable healthcare patient service systems to share and use data between system components more effectively. The techniques can allow for devices often found within hospitals and other healthcare facilities to better operate with one another and allow more data from various third party devises and systems to be accessed, stored, and analyzed for more informed patient care decisions to be made while also allowing the patient and caretakers more control over patient care.

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

With the increasing amount of IoT devices in all settings, including healthcare settings such as hospital hallways, patient rooms, and surgical rooms, there is a need for systems that allow for the devices to effectively be incorporated into existing and future hospital systems. Further, a solution for such a need would also benefit from being able to ensure that various devices and systems within healthcare settings are able to communicate with the other devices and systems in healthcare settings. One such system, commonly seen in a hospital setting, is a nurse call system. There are various existing nurse call systems on the market, each with its own functionalities, required inputs, available outputs, and communication protocol requirements. Thus, there is an additional need in the field to allow various devices in healthcare settings to effectively communicate and work in tandem with various possible nurse call systems.

Examples herein are directed to among other things, systems and methods relating to using a hub to allow devices in a healthcare setting to communicate with a nurse call system, and vice-versa.

Some examples herein are directed to among other things, systems and methods relating to using a hub to allow devices in a healthcare setting to communicate with other devices and/or systems in a healthcare setting.

In an example system, a hub and a server are communicatively coupled. The server may be configured to receive a first request of a device associated with the room, the first request associated with at least one of the room or a patient in the room. The first request may contain information to be sent to the nurse call system. The server may determine what permissions should be associated with the first request by determining that at least one of a user of the device or the device itself is permitted to make the first request. The server may also use the determined permission to further determine the room associated with the first request and the hub associated with the room. The server may then send a second request corresponding to the first request to the determined hub. The hub may then receive the second request from the server, generate, based on the nurse call system, a third request that corresponds to the second request and that comprises the information from the first request, and transmit the third request to the nurse call system.

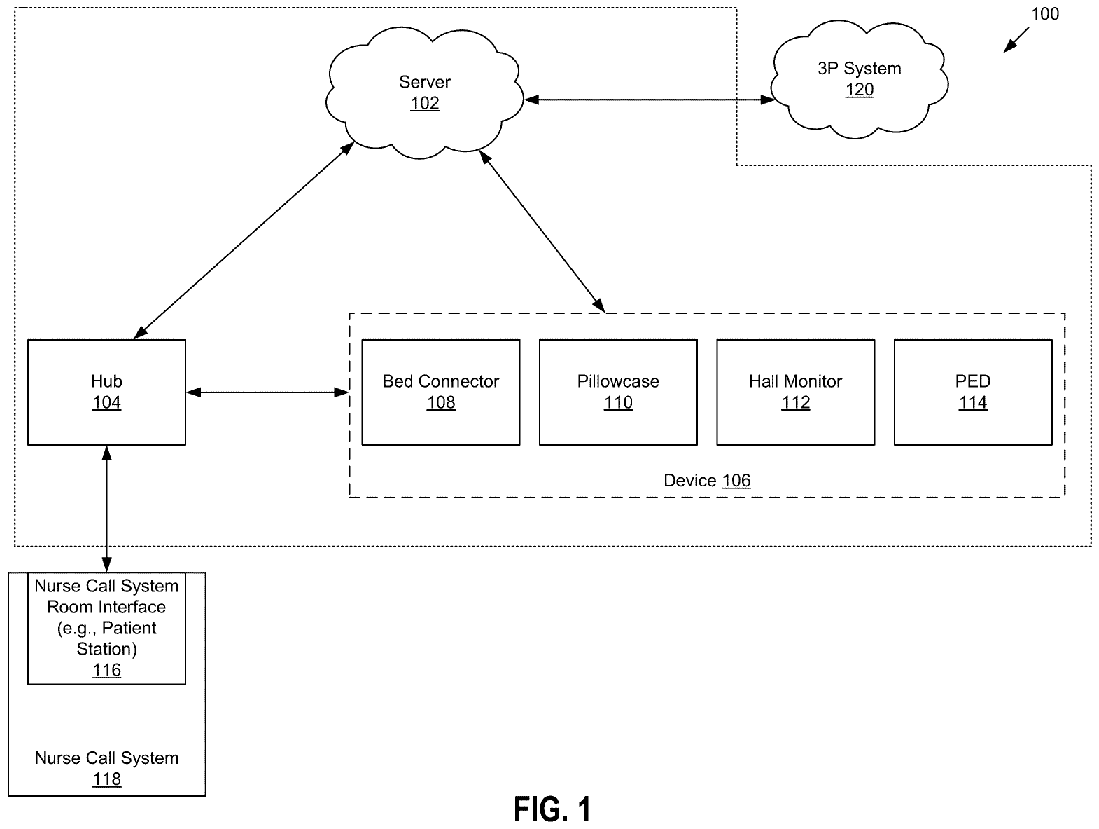

Turning now to the figures, FIG. 1 illustrates a block diagram of a system according to an embodiment. The system in FIG. 1 includes a server 102, a hub 104, a third party system 120, a nurse call system room interface 116, a nurse call system 118, and one or more devices 106.

The hub 104 can be configured as an intermediary component between the server 102 and device(s) 106, the device(s) 106 and the nurse call system room interface 116, and/or the server 102 and the nurse call system room interface 116.

In some embodiments, the hub 104 may not need to generate or pass along requests in response to the requests it receives. The hub 104 may be the destination for requests originating from system components. For example, a first device 106 may send first device 106 data to the hub 104 that corresponds to the first device's 106 current state so that it can be retrieved at a later point in time by a second device 106 while only needing to query the hub 104 for the first device's 106 state. In an embodiment, the hub 104 may be sent permissions information to associate with a device ID and be requested to store the association.

The hub 104 may be used to provide passive data passthrough functionality or may actively modify data before sending it on to the next component in the system.

In one example, the hub 104 includes multiple hardware modules. A first hardware module can provide data connectivity. This same hardware module or a different hardware module can also provide power (e.g., this module can be a POE module). A second hardware module can provide data translation functionality between the nurse call system and the one or more devices. The data translation functionality can involve re-formatting data (e.g., using a particular serial format) sent from a device 106 to the nurse call system room interface in a format that is usable by the nurse call system room interface 116. Conversely, the data translation functionality can involve re-formatting data (e.g., using a particular serial format) sent from the nurse call system room interface 116 to a device 106 in a format that is usable by the device. These two functions of the data translation functionality can be implemented via specialized hardware of the second hardware module and/or software executing on general-purpose hardware of the second hardware module. In particular, depending on the type and/or model of the nurse call system 118, the proper specialized hardware or the proper software can be installed in the hub 104. The hub 104 can include one or more processors and one or more memory storing instructions that, upon execution by the one or more processors, configure the hub 104 to control the two hardware modules and/or provide the data connectivity and/or the data translation functionality. Alternatively, rather than using multiple hardware modules, a single hardware module may be used.

Further, the hub 104 might also modify the data based on a determination of whether certain conditions have been satisfied before sending the data. Conditions may be obtained by the hub 104 from device(s) 106, the server 102, local storage, and/or the nurse call system room interface 116. For example, conditions that the hub 104 may evaluate are whether a bed rail is down, whether a bed is powered, whether a cord has become detached (e.g., based on voltage levels, time since last communication, etc.), whether the patient has pressed a call button, how much time has passed since a particular event, etc. The hub 104 may also use a patient identifier (ID), a Portable Electronic Device (PED) ID, a user ID, a bed ID, a room identifier (room ID) and/or a hub ID when making modifying and generating requests. In one example, the hub 104 and/or device 106 uses a MAC address as its unique ID, or stores an assigned ID in non-volatile memory. In one example, the hub 104 can receive any of the IDs and provide the received ID(s) to any one of the one or more devices 106 via the data connection with the one or more devices 106. Any of the components or users of the components within the various possible embodiments may contain a unique ID to be stored in a relational database.

The hub 104 may associate the different IDs by storing them in one or more data structures (e.g., databases, tables, etc.) during provisioning of the room, when receiving requests, upon a check in/check out of a patient (e.g., using electronic medical records), upon the hub 104 being installed in a room, upon a device 106 being installed in the room, and/or during any other time. For instance, during the installation process of the hub 104 in the room, the association between its hub ID, the bed ID, and the room ID can be generated and stored. In an embodiment, device 106 ID's (e.g., hall monitor 112 ID, PED 114 ID, pillowcase 110 ID) may be associated with a hub 104 ID (MAC address), and the hub 104 ID is associated with a user ID. In an embodiment, during a check in process of the patient, the association between the patient ID and the PED ID, the user ID, the bed ID, the room ID, and/or the hub ID may be generated and stored. During a check out process, some, or all, of these associations between IDs may be removed (e.g., deleted from the data structure). In some embodiments it may be helpful to retain stored IDs and related information after a patient is checked out so that user preferences can be saved in case a user or patient uses the same systems and/or devices at a later point in time. Further, the stored IDs and related information may be helpful to keep after a patient is checker out, such as in the case that the stored IDs are for non-patient devices and should retain at least some of their associations (e.g., associations between a device 106 that is in the same room as a hub 104).

Associations between IDs of devices 106, hubs 104, rooms, patients, caregivers, and permissions may be based on many factors. For example, in addition to or alternatively to any other ways of association that are discussed, associations of IDs and/or permissions may also be based on a user being within a proximity of a hub, a beacon device, at a certain GPS location, another user, and/or any combination thereof. Associations (between system components, users, and/or permissions) may be made automatically, such as based on location or may be made manually, such as based on user unput, or a combination thereof.

The association of IDs may be useful for generating, sending, and/or relaying requests between system components. For example, a user ID may be useful in associating the actions of a user ID with a certain room ID so that a user may perform actions relating to a particular room. In an example, knowing that a device ID is in communication with a certain hub or server may allow for the device to be associated with a room, care center, person, etc.

Permissions related to any of the IDs may also be granted and/or stored during provisioning of the room, when receiving requests, upon a check in/check out of a patient (e.g., using electronic medical records), upon being installed in a room, when a condition has been satisfied (e.g., the system may update permissions upon the condition that it is a certain time of day or a patient's status is changed), and/or during any other time. Permissions related to any of the IDs interacting within the system allow various permission levels to be configured for the fine-grain control of actions that can be taken by certain rooms, devices, people, etc. In an embodiment, nurses, users, and system administrators have the ability to grant certain permissions. In an embodiment, a user can grant temporary or permanent permissions to give room control ability to a relative, friend, spouse, etc. In an embodiment, permissions may change based on certain conditions or events such as the time of day, for example, a television patient room device may not be able to exceed a certain volume past a certain time of day. Various possibilities exist to utilize the permissions and IDs associated with the people and components of the systems described herein.

As an example of how permission may work in an embodiment, a patient may have a user ID. The user ID may be associated with a device 106 (e.g., one of their personal devices such as a mobile device) having a device ID. Further, the patient's user ID may be associated with a room ID of the room the patient is staying in. The association between the user ID, device ID, and room ID may be stored by the server 102. The patient may then use their device 106 with requisite permissions to perform actions related to the room (e.g., make a nurse call, change the TV station, adjust the bed height, adjust the room temperature, adjust the lighting, etc.). As the user performs actions related to the room, requests may be sent to the hub and/or server 102 to determine if the user and/or device 106 have the requisite permissions to perform the actions. In one embodiment, the hub 104 receives a request from a device 106, the hub 104 then queries the server 102 (or vice versa) regarding the obtainment of permissions-related data before the hub 104 assesses using the obtained permissions-related data to determine whether the requisite permissions are granted to the user and/or device 106. Alternatively, the hub 104 may receive a request and then send a second request to the server 102 (or vise-versa) corresponding to the request received by the hub 104 so that the server 102 can determine if requisite permissions for the action are satisfied.

It would be obvious to one of skill in the art that as a patient, nurse, doctor, visitor, or other person moves from room to room, the permissions and preferences associated with their user ID would follow them. Thus, it would be possible for a patient to be assigned to a new room on a different floor of a facility, and once they are brought there, their current preferences allow for the shades, televisions, lights, and other devices to behave in the same way as their prior room based on stored preferences associated with the patient. Further, a similar patient movement scenario may result in devices in the new room adjusting to match their last known state in the prior room (e.g., the shades were closed in the prior room and once the patient is moved to a new room, the shades close to match the shade state to the prior room). In such a patient movement scenario, the permissions may additionally, or alternatively, change to grant the patient's user ID, device ID, or other identifier the ability to successfully interact with the system components associated with the patient's new room and take away permissions related to the prior room (e.g., the patient can control the lights in their new room and can no longer do so in their prior room). An example of how a patient's permissions could change as they are brought into a new room could be based on whether the patient's user ID has permissions and/or an association with the hub 104 that is in the new room.

The hub 104 may store the various identifiers (e.g., PED ID, user ID, etc.), associations, preferences, permissions associated with the various identifiers, permission evaluation logic, or any other data it uses in data structures locally within the hub, remotely (e.g., server 102), or in a remote/local combination (e.g., duplicate the local data to a remote server, store certain data locally and other data remotely, etc.). Further, the server 102 may alternatively or redundantly store some or all of the information that could be stored by the hub 104. In an embodiment, the hub 104 downloads, from the server 102, and then locally stores, permissions associated with a user ID upon a patient being checked in and associated with the hub 104. In an embodiment, every time the hub 104 receives a request and needs to check if related permissions are present, the hub 104 sends a request to the server 102 so that the server can evaluate if the request received at the hub 104 satisfies the necessary permissions threshold.

The hub 104 might be installed in the wall, on the surface of the wall, on the bed, or positioned within the room, among other places. Further, the hub 104 might also contain a screen (e.g., a touchscreen), physical button and/or switches (e.g., physical buttons), barcode, QR code, or other ways of interfacing with a user or user device (e.g., tablet, phone) so that hub 104 configurations can be made. Example hub configuration possibilities can be which one or more devices 106 are to be connected to (e.g., when installing a new device 106 in the room to communicate with the hub 104, when adding a user and/or user device to control devices in the room, etc.), whether a device interface board is being used, whether an input combination board is being used, whether a certain type of nurse call translation board is being used, whether a software update should be installed to components of the hub 104, etc.

In an embodiment the hub has one or more lights and/or speakers. Lights and speakers may be useful for communicating with a user and/or a device. Light may display images and/or colors on the ceiling or wall. Further, lights may also be capable of flashing or displaying certain colors that indicate a certain meaning.

The nurse call system 118 represents a nurse call system that is being used in a healthcare setting. Various types of nurse call systems may be used in such a setting and can be available from one or more nurse call system providers. The nurse call system 118 may be running on a computer local to the hospital or may be running on a remote server. The nurse call system 118 is capable of receiving information from the nurse call system room interface 116 that originated from other components in the system (e.g., the server 102, the hub 104, the third party system (3P system) 120, a patient room device 106, etc.). The nurse call system 118 is also capable of sending information to other components in the system (e.g., the server 102, the hub 104, the 3P system 120, a patient room device 106, etc.) via the nurse call system room interface 116. As an example, the nurse call system 118 may receive a request via the nurse call system room interface 116 (e.g., a patient station), perform a nurse call system action, and send a request to a patient room device 106 to perform a certain action. In an embodiment, the nurse call system may also send and receive requests to/from other components in the system directly, without the use of the nurse call system room interface. In an embodiment, the nurse call system interface is included within the hub thereby allowing the hub to be directly (wired or wirelessly) connected with the nurse call system.

The nurse call system room interface 116 can be installed in the room, but need not be. For instance, the nurse call system room interface 116 may be a patient station in the patient room. The hub 104 can be connected thereto via a wired connection (e.g., 1/4 connector, a multi-pin connector according to a proprietary design or a standard such as a 37-pin connector, a USB connector, or other similar connector), etc.), or a wireless connection (e.g., a radio-frequency-based connection, magnetic-based connection, optical-based connection, etc.). The nurse call system room interface 116 is one way to allow for the effective communicative coupling of the hub 104 and the nurse call system 118. The nurse call system room interface 116 can allow for communications to the nurse call system 118 from the hub 104 and/or server 102, and from the nurse call system 118 to the hub 104 and/or server 102. As another example, the nurse call system room interface 116 may be outside of the room in the hallway within reach of a wired or wireless connection from the hub 104. In an embodiment, the nurse call system 118 may use wired or wireless communication to send and receive requests to/from the server 102 without the use of the nurse call system room interface 116.

The server 102 can be configured as an intermediary component between the hub 104 and the one or more devices 106, between the 3P system 120 and the hub 104, between the 3P system 120 and the devices 106 between the nurse call system 118 and the hub 104, between the nurse call system 118 and the 39 system 120, and/or between the nurse call system 118 and one or more devices 106. One or both of the data translation functions discussed above in relation to the hub 104 can be implemented by the server 102 in addition to or as an alternative to the data translation functions of the hub 104. The server 102 may be used to passively relay requests toward the destination component or may actively modify data before sending it on to the next component in the system. In an embodiment, the destination may be determined based on logic at the server 102 and/or hub 104. In an embodiment, the logic to determine where to send the message may observe the message content, a sending component (e.g., device 106. hub 104, etc.) identifier, a destination device identifier, the message type (e.g., emergency request message), or other information associated with the message. Thus, in some embodiments, it is possible that a message is sent to the hub 104 and/or server 102 and the message does not have another destination component, but may then be sent to an appropriate destination component based on logic carried out by the hub 104 and/or server 102. Further, in some embodiments, the server 102 may modify the data in order to translate the information sent by a prior component into a form usable by a subsequent component. Further, the server 102 might also modify the data based on a determination of whether certain logic conditions have been satisfied. Condition parameters may be obtained by the server 102 from device(s) 106, the hub 104, local storage, and/or a third party system 120. Data used for evaluating conditions at the server 102 side may be similar to the data used at the hub 104 for evaluating conditions.

In some embodiments the server may also store logs of all incoming requests, outgoing requests, and server processing that takes place. In some embodiments, the server may use stored log data to generate information for patients, doctors, nurses, system administrators, architects, and other personnel. In an embodiment, the stored data may be used by other system components and/or 3P systems (e.g., machine learning systems that can be used to control the graphical user interfaces (GUIs) of different devices that may use the log data as training data for dynamic GUI models). In an embodiment, the server only collects log information corresponding to certain user IDs that have granted such a collection permission. One of skill in the art would recognize other ways to control what information is logged and what can be done with the information.

The server 102 may use a patient ID, a PED ID, a user ID, a bed ID, a room ID a hub ID, and/or other identifiers when making modifying and generating requests. The server 102 may associate the different IDs by storing them in one or more data structures (e.g., databases, tables, etc.) during provisioning of the room, when receiving requests, and/or upon a check in/check out of a patient, and/or during other times. For instance, during the installation process of the hub 104 in the room, the association between its ID and the bed ID and the room ID can be generated and stored. During a check in process of the patient, the association between the patient ID and the PED ID, the user ID, the bed ID, the room ID, and/or the hub ID can be generated and stored. During a check out process, this association can be removed (e.g., deleted from the data structure). Other ways in which storage of IDs, storing relationships among IDs, storing permissions, storing preferences, evaluating conditions, and evaluating permissions that were discussed above may be implemented on the server 102 in addition to, or as an alternative to, the hub 104.

In some embodiments, the server 102 may not need to generate or pass along requests in response to the requests it receives. The server 102 may be the destination for requests originating from system components. For example, a first device 106 may send first device 106 data to the server 102 that corresponds to the first device's 106 current state so that it can be retrieved at a later point in time by a second device 106 while only needing to query the server 102 for the first device's 106 state. In an embodiment, the server 102 may be sent permissions information to associate with a device ID and be requested to store the association.

In an embodiment, the server is a server running on a cloud platform and/or is on premises. The server may be accessible by only a local network, in an embodiment.

As illustrated, there may be a data connection between the server 102 and the hub 104, the server 102 and device 106, and the server 102 and the 3P system 120. For instance, each of the data connections can be implemented over a public (e.g., the internet) or private network (e.g., an intranet), whereby an access point, a router, and/or another network node can communicatively couple the server 102 to the hub 104. Data connection between the components can be a wired data connection (e.g., 1/4 connector, a multi-pin connector according to a proprietary design or a standard such as a 37-pin connector, a USB connector, or other similar connector), etc.), or a wireless connection (e.g., a radio-frequency-based connection, magnetic-based connection, optical-based connection, etc.). Data connections may also be made through the use of a mesh network. For example, more than one hub 104 may be communicatively coupled with one another hub in order to communicate with a server 102 or other system component. Similarly, a mesh network may be used among devices in order to communicate with a hub and/or server. Further, data connections exist between other system components, such as the data connections between the hub 104 and nurse call system room interface 116, between the hub 104 and device 106, and between the nurse call system room interface 116 and the nurse call system 118. A data connection between a device 106 and the hub can additionally, or alternatively, be a power connection. A power connection can supply power to the connected component via the hub 104 or vice versa. The data connection can provide for data moving to and from device 106 to the hub 104. For instance, the connection between device 106 and the hub 104 can include a wired AC or DC power connection (or a magnetic connection for wireless charging) and a Bluetooth connection (or some other communication protocol, RF, optical, or otherwise). In another illustration, the connection between the patient room device 106 and the hub 104 can be a power-over-Ethernet (POE) connection. One having ordinary skill in the art would recognize that components of the systems described herein may be communicatively coupled through the use of a network (e.g. a LAN, WAN. Etc.). Further components of the systems described herein may be communicatively coupled through a combination of wired and wireless means (e.g., wireless connection to a router than is connected via an ethernet cable to a server).

The interfaces between the system and components communicatively coupled with the system, as well as interfaces between the components within the system, can be implemented using application programming interface (APIs). For example, the server 102 can implement a set of APIs for communications with the 3P system 120. Similarly, the hub 104 may, but need not, implement a set of APIs for communications with the nurse call system 118 via the nurse call system room interface 116. For another example, the server 102 can implement a set of APIs for communications with the hub 104 and for communications with device 106. Additionally, or alternatively, the hub 104 and/or a device 106 can implement a set of APIs for communications with each other or with the server 102. As a further example, a server 102 may implement a set of APIs for communications with the nurse call system 118 (not being routed through the nurse call system room interface 116).

The third party system 120 can include one or more servers and/or one or more devices of a third-party for third-party related functionality available to the hospital and/or to patients of the hospital. One example of such 3P system 120 may be an HVAC system run by a third party. In an embodiment, the 3P system 120 is associated with a healthcare company. In an embodiment, the 3P system 120 is associated with the nurse call system 118.

The device 106 is one or more devices (e.g., a bed connector 108, a pillowcase 110, a hall monitor 112, a PED 114, a camera, a lamp, shades, etc.). Any one of the one or more devices 106 may be installed in the room as in in-room device and/or may be outside of the room as an out-of-room-device. Further, a device 106 may sometimes be an in-room device and sometimes be an out-of-room device as it gets moved to and from the room. A device 106 that is partially or fully located within a wall, ceiling, and/or floor of a room may also be considered to be an in-room device. Any one of the one or more devices 106 may be referred to as a patient device, user-device, medical device, care team device, and/or a registered device, etc. Such device 106 labels may be able to identify the type of device 106 (e.g., smartphone, smartphone of patient, tablet of care team, hospital bed, pump, etc.) and are not exclusive labels. Thus, a device 106 may be classified as a medical device and simultaneously classified as a care tram device. A device may move in and out of the building. Additionally, a device 106 may be in the possession of, for example, a patient, nurse, friend of patient, relative of patient, etc. Therefore, a device 106 is not necessarily a device controlled by a patient and can be controlled by any user or may be configured to be controlled only by authorized users. Further, a device may also not be in the possession of anyone person (e.g., lamp, bed, room camera, etc. that is placed in a room). A device 106 may also be a medical device (e.g., heart rate monitor), a non-medical device (e.g., personal phone, hospital owned tablet, lamp, etc.), etc. A device 106 may or may not be registered and/or certified with the FDA. As seen, many ways to classify the devices that are operating as part of the system exist, all the ways of which have not been explicitly described. A device 106 may be communicatively coupled to the hub 104, server 102, and/or another device 106 directly or indirectly (e.g., through a hub 104, server 102, other device 106).

The bed connector 108 can be installed in a bed located in the room and can provide power and/or data connectivity to the bed in support of bed related functionalities. These functionalities can relate to bed controls and bed data (e.g., whether the bed is being used, the position of the bed, etc.). The bed may have an identifier (e.g., a bed ID). The server 102 can associate the bed ID with the room ID and/or the hub ID. The bed connector is an example of an in-room device.

The pillowcase 110 can also be installed in the bed and can allow pillowcase 110 related functionality to a patient that may be using the bed (e.g., nurse call assistance requests, room controls, etc.). The power may be supplied to the pillowcase 110 via the connection to the hub 104 or may come from a different power source. The data connectivity can be provided via the connection with the hub 104 and/or the data connection with the server 102. The pillowcase 110 may have an identifier (e.g., a pillowcase ID). Server 102 and/or hub may associate the pillowcase ID with the bed ID, the room ID, and/or the hub 104 ID. The pillowcase device is an example of an in-room device.

The hall monitor 112 can be installed outside of the room such as on a wall facing a hall next to the room and close to an entry door of the room. The hall monitor 112 would be an example of an out-of-room device. The hall monitor 112 may be connected to the hub 104 directly or to be connected to the hub indirectly via the server 102. The hall monitor 112 can provide monitoring and information functionality related to the patient and/or the room. This functionality can include displaying to a nurse (or any other authorized user) information about the patient and/or about the occupancy of the room or the bed. Thus, the hall monitor 112 is an example of a device capable of being used by a nurse, or other authorized user. This functionality can also include enabling two-way audio and/or video communication with a device located in the room (e.g., the PED 114 when such a device is in the room and operated by the patient). Power and data connectivity can be provided to the hall monitor 112. The power may be supplied via the connection between the hub 104 and the hall monitor 112. The data connectivity can be provided via the connection with the hub 104 and/or the data connection with the server 102. The hall monitor 112 may have an identifier (e.g., a hall monitor ID). The server 102 may associate the hall monitor ID with the room ID and/or the hub ID.

The PED 114 can be located inside or outside the room. Thus, PED 114 is an example of a device that can sometimes be an in-room device and sometimes be an out-of-room device. In one example, the PED 114 can be a mobile device (e.g., a smart phone, a tablet, etc.), the mobile device may be securely attached or releasably attached to the bed and available to the patient. In an embodiment, the PED 114 can provide pillowcase related functionality and/or other patient related functionality (e.g., to stream video content, to play music, to check out of the room/hospital, to perform audio/video calls). For instance, the PED 114 includes one or more program codes executable thereon to provide any of these features. Power and data connectivity can be provided to the PED 114. The power may be supplied via the connection with the hub 104. In comparison, the data connectivity can be provided via the data connection with other devices 106, the hub 104 and/or the data connection with the server 102. The PED may have an identifier (e.g., a PED ID). The server 102 can associate the PED ID with the bed ID, the room ID, and/or the hub ID.

Alternatively, the PED 114 can be located outside the room and can be operated by an authorized user. Therefore, the PED 114 is an example of a device that can be operated by a patient or non-patient. The authorized user can be, for instance, a hospital personnel (e.g., a nurse, a doctor, etc.) or a person that the patient authorizes to have access to the patient when in the room and/or to have access to room functionalities (e.g., temperature control), and/or a device (e.g., a camera that can be monitoring the patient and/or the room). In the case of a hospital personnel, the PED 114 can provide pillowcase related functionality and/or other hospital functionality (e.g., monitor the patient, receive nurse call requests thereof, etc.). In the case of a patient-authorized person (e.g., a family member, a friend, etc.), the PED 114 can provide communication and monitoring functionality (e.g., to call the patient, to receive patient data (e.g., health update, sleep status, etc.). In the case of an authorized device, the PED 114 can provide monitoring and alert functionality (e.g., a camera with an artificial intelligence that processes the images to detect whether the patient is in the bed or has fallen off, respiratory rate, etc. and generate alerts (e.g., notifications, alarms, etc.) as needed). For instance, the PED 114 may include one or more program codes executable thereon to provide any of these features. Because the PED 114 may be located outside of the room, data connectivity thereto may not be provided via the connection with the hub 104 and, instead, may be provided via the data connection to the server 102. Here also, the PED 114 may have an identifier (e.g., a PED ID) and the authorized user may have an identifier (e.g., a user ID). The server may associate the PED ID with user ID, the room ID, and/or the hub ID. On with ordinary skill in the art would recognize that the PED ID (and other identifiers) may be associated with any other device identifiers, users, rooms, floor levels, departments, locations, permissions, etc.

Although a single PED 114 is illustrated in the figure, multiple PEDs can be used, some of which may be located inside the room and other ones may be located outside the room. The PED 114 or other patient room devices 106 may have a GUI that is configurable by the user based on the devices 106 (or other users, rooms, etc.) associated with the room, user, etc. For example, a user may be able to add or move controls on a tablet screen GUI for shades if their room has shades. In an example, a user may not have the option to configure any shade-related GUI if they are not associated with a room that has shades. For example, a user PED 114 may allow a user to control the sunshades in a room that is associated with the user and/or device of which the user and/or device has permission to control. If certain rooms have a different number of shades, the GUI on the PED 114 could allow the user to visually determine how many shades they can control and the open/closed state of each of the shades. In a second example, a PED 114 that is located outside of the room may have less permitted control ability than a PED that is located within the room on the basis of where it is located and therefore the dynamic GUI of the PED 114 would reflect the lesser degree of control ability than if the PED 114 was in the room. Such functionality may be obtained by dynamically changing permissions based on where a device is, or merely changing the GUI based on device location, even if permissions are granted, among other methods. In an embodiment, such changes in control ability may be based on whether the PED 114 is sending requests directly between the PED 114 and hub 104, whether the PED 114 is communicating with the hub 104 via the server 102, whether the device is at a certain location or not, whether a certain hub 104 is being used by a PED 114 (or other device 106) to relay a request, etc. In yet another embodiment, whether the PED 114 is sending requests directly between the PED 114 and hub 104 or whether the PED 114 is communicating with the hub 104 via the server 102 can be used to configure the GUI of a PED 114, regardless of the permissions associated with the PED 114, user, etc.

A PED 114 might also have a certain GUI with fewer displayed items if a user has fewer permissions. In one embodiment, a user ID may have permission to adjust an associated room's temperature but not adjust the bed height. In such an example, the GUI of the user's device 106 associated with the user ID may not show the bed height adjustment controls because they do not have the requisite permissions. Alternatively, the GUI (or other user interface, such as a bedside remote controller) may show the bed controls even if the user ID associated with the user's device 106 does not have the requisite permissions, but the request from the user's device 106 may be disregarded by the hub 104 and/or server 102 after the hub and/or server 102 determines there are no requisite permissions to perform such action. A GUI may further change based on the patient due to past preferences of the patient that have been stored, such as in storage on server 102. Other room-based, user-based, permissions-based, location-based, or the like GUI customizations could be implemented.

In an example, the hub 104 is installed in a room of a hospital. The server stores an association between an identifier of the room (e.g., a room ID) and an identifier of the hub 104 (e.g., a hub ID). The server 102 need not be installed in the room and may even be remote from a hospital location (e.g., by being a cloud-based server). Any one of the one or more devices 106 may be installed in the room and/or may be outside of the room. Continuing the example, a pillowcase 110 might receive a user interaction asking for nurse assistance. In this example, the pillowcase 110 may then send a first request from the pillowcase 110 corresponding to the user assistance request to the hub 104. The hub 104 might then translate the first request received from the pillowcase 110 into a second request format, that is capable of being successfully interpreted by the nurse call system room interface 116, and then send the second request to the nurse call system room interface 116 which would then communicate with the nurse call system 118 to subsequently inform a nurse that the patient who caused the pillowcase 110 event has requested assistance.

In a different example, the hub 104 may receive the assistance request message from the pillowcase 110, use locally (e.g., hub 104) or remotely (e.g., remote server 102, local on-premises server 102, etc.) stored data (e.g., configuration information, permissions information, and/or data received from devices or other system components, etc.) to further inform the hub 104 what to do next, and then send a first assistance request to the nurse call system room interface 116 according to the decision that was made using a combination of (locally or remotely) stored data and the first assistance request. The second request to the nurse call system room interface 116 may correspond to the first assistance request or may have been changed. One change that might occur to a message is changing the assistance request into an emergency call. For example, an assistance request might be changed by the hub 104 to an emergency call if a device 106 in the room of the patient, such as a camera in the room, has also sent data to the hub 104 that allow the hub 104 to infer that an emergency request should be sent. In such an example, a camera in the patient's room may recognize, possibly through image recognition, that the patient has fallen, so the hub 104 will take the fall determination and the assistance request message from the pillowcase 110 to determine an emergency call should be made, and then generate and send an emergency call request to the nurse call system room interface 116. In the above example, the server 102 may perform similar functions to the hub 104 in addition to or as an alternative to the hub 104 performing the functions.

The opposite flow of information can be followed, where a request can be sent from the nurse call system 118 to one or more devices 106. This request can be first routed via the nurse call system room interface 116 to the hub 104, reformatted by the hub 104 as applicable, sent from the hub 104 to the server 102, and then sent from the server 102 (and reformatted as needed) to the one or more devices 106. Other information flows also exist such as sending a request from the nurse call system 118 to one or more devices 106 by routing a first request from the nurse call system 118 to the server 102, reformatting the first request by the server 102 as applicable, sending a second request from the server 102, corresponding to the first request received by the server 102, to one or more device 106.

Similar data flows can also be followed when the third party system is involved, whereby a request is passed from the third party system to the server or vice versa.

Various other information flow possibilities exist with the disclosed systems and methods.

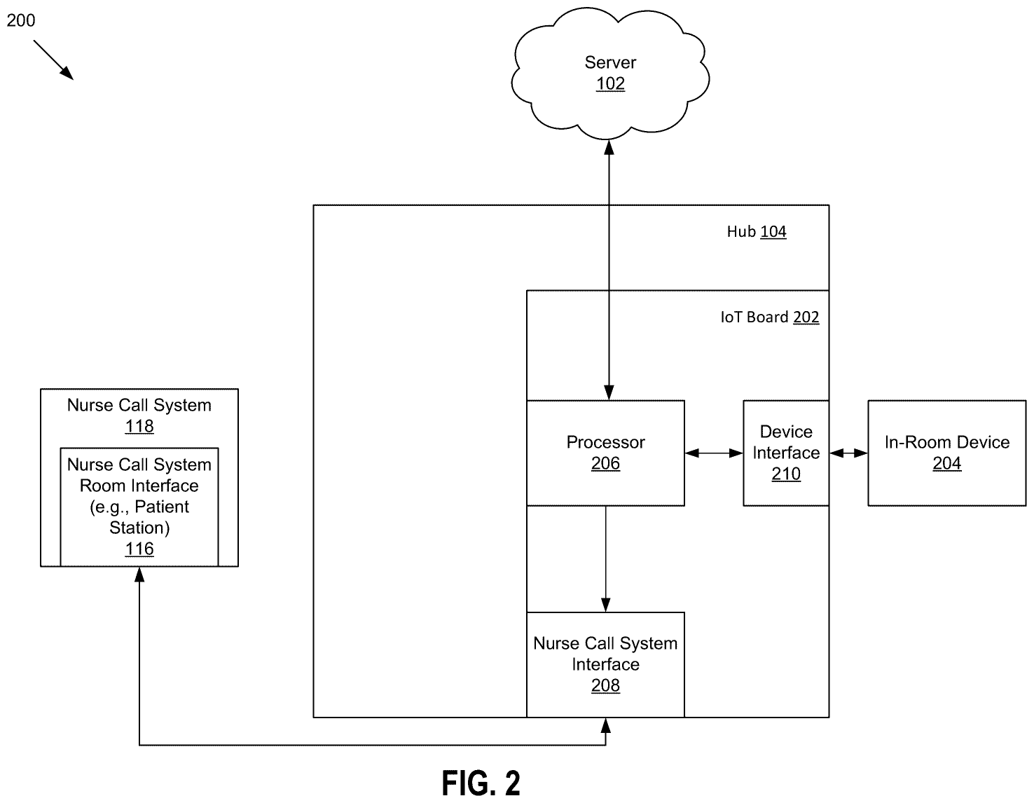

Turning now FIG. 2 which illustrates a block diagram of a system according to an embodiment. The system 200 includes a hub 104, an IoT board 202, a processor 206, an in-room device 204, a device interface 210, a nurse call system interface 208, a nurse call system room interface 116, and a nurse call system 118, and a server 102. Notably, in this example embodiment, the nurse call system interface 208 supports two-way communication.

The nurse call system room interface 116 and the nurse call system 118 function similarly to how they were already described concerning system 100.

Similarly, the hub 104 has also been described in relation to system 100. However, components that may make up the hub 104 are further depicted in system 200. The components depicted to be within the hub 104 in FIGS. 2-5 may be contained within the housing of the hub 104, may be external to the hub 104 housing, or might be a combination of externally and internally housed from/within the hub 104. Further, certain components may be hot-swappable so that they can be changed on the fly without the need to buy a second hub 104 to obtain different possible component configurations. Certain components may also be configurable to allow for different functionality without the need to buy a second hub 104 to achieve the desired functionality.

The hub 104 may include a processor 206 for executing instructions and memory (not shown). The hub may be capable of receiving requests. The processor 206 may translate requests received from various other system components before sending a subsequent request to another system component. The processor 206 may not need to translate the request format before sending a subsequent request to a system component. Additionally, the processor may use data from one or more system components as inputs to determine and generate a corresponding output to send to a system component. The processor 206 may receive data input from device(s) 106 (e.g., an in-room device 204) via a device interface 210. The processor 206 may be configured to be communicatively coupled with a server 102. The processor may output data to the nurse call system interface 208. Further, the processor 206 can output data to device(s) 106 (e.g., an in-room device 204) via a device interface 210. The processor 206 may also output data to the server 102. In an embodiment the processor might not only translate, alter content of, and/or pass along requests but it might, alternatively or additionally, be the originator of requests which are subsequently sent to device(s) 106 (e.g., in-room device 204), nurse call system room interface 116, server 102, a third part system, or a nurse call system 118 directly, or indirectly through another system component. As an example, the processor may be in the form of an ESP32 chip.

Although, for simplicity, the descriptions contained herein describe the processor 206 as being communicatively coupled with the server 102 or other components, and the figures display a connection with the processor 206 component, it would be understood by one of skill in the art that the loT board 202 facilities such connectivity. The connectivity between the processor 206 and other system components may be facilitated by a separate chip on the IoT board 202 that enables Bluetooth, Wi-Fi, 5G, Zigbee, or other wireless communications. Further, the connectivity between the processor 206 and other system components may be achieved through a wired connection (e.g., ethernet cable). One of ordinary skill in the art would recognize that there are many ways in which a processor 206 on a IoT board 202 can be configured to be in wired and wireless communication with other components attached to and not attached to the same IoT board 202.

The hub 104 can be powered from a power source. The power source can be internal to the hub 104 (e.g., a rechargeable battery, a disposable battery, etc.) or external to the hub 104 (e.g., from a wall power outlet). If external, the power can be provided via a cable (e.g., a dedicated power cable, a multi-functional cable such as a power-over-ethernet compatible cable) or wireless (e.g., via magnetic charging). The power to the hub 104 is used to power the components depicted within the hub 104 and any additional or alternative components that could be incorporated into the hub 104. The hub may also be used to provide temporary or permanent power for other system components (e.g., PED, pillowcase, hall monitor, etc.). As an example, the hub may provide power to a PED via a G14 cable. The in-room device 204 may obtain power from the hub 104 using a power over Ethernet unit configured to provide power to the in-room device 204 via a cable that couples the hub 104 and the in-room device 204 and to provide the in-room device 204 with data connectivity to a data network via the cable or via wireless communication path between the hub 104 and the in-room device 204. The hub 104 may power various kinds of devices (e.g., patient devices, non-patient devices, in-room devices, out-of-room devices, etc.).

The in-room device 204 may be a device that allows for user input (e.g., PED). The in-room device 204 may be in the form of a pillowcase controller. The in-room device 204 may be communicatively coupled with the processor 206 of the hub 104 via a device interface 210 so that user input and other data generated at the in-room device 204 can be sent to the processor 206 of the hub 104. The in-room device 204 may additionally receive information from the processor 206 via the device interface 210. In an embodiment, in-room device 204 may be communicatively coupled with the server 102.

The nurse call system interface 208 can be used as an interface between the processor 206 and the nurse call system room interface 116. Although system 200 depicts nurse call system interface 208 as supporting two-way communication between the nurse call system interface 208 and the nurse call system room interface 116, in some embodiments the nurse call system interface 208 only supports one-way communication from the nurse call system interface 208 to the nurse call system room interface 116. The nurse call system interface may send (and in some embodiments, additionally receive) requests to (and in some embodiments, additionally receive requests from) the nurse call system 118 via the nurse call system room interface 116 (e.g., nurse call, emergency nurse call, channel change request, shade movement request, etc.). The request output from the nurse call system interface 208 may not require any additional data formatting from the point in which it is output from the processor 206 before the nurse call system room interface 116 is able to correctly interpret the meaning of the message. Therefore, for an output from the nurse call system interface 208 may be used to reduce the amount of processing that the message would need to propagate through before reaching the nurse call system 118 compared to other possible embodiments. This can allow for a request to be received by the nurse call system 118 more quickly and may also allow for less chance for error to be introduced into the signals of the request. In an embodiment, the nurse call system interface 208 communicatively couples to the nurse call system room interface 116 with the use of a wired ¼ inch connector.

The IoT board 202 contains the processor 206, a device interface 210, and a nurse call system interface 208. Although system 200 depicts the processor 206, device interface 210, and nurse call system interface 208 as being within the IoT board 202, these components may be housed in or on an IoT board 202. Further, the components may not be on a physical board, but may be coupled with the IoT board 202.

As an example of a system 200, consider a system 200 where the hub 104 is powered by a single wired dedicated power source. A patient in a hospital setting may press a button on a nurse call pillow in-room device 204 that signifies the patient would like the TV in their room turned on. The in-room device 204 would send a request to the processor 206 via the device interface 210. The processor 206 could then send a corresponding request via the nurse call system interface 208 to the nurse call system room interface 116. The nurse call system room interface 116 would then use the received request to prompt the nurse call system 118 to alert a nurse to the initial request made by the patient at the in-room device 204.

The server 102 in example system 200 is illustrated as being situated similarly to the server 102 in system 100. The server 102 in system 200 is communicatively coupled to a processor 206. The server 102 may be configured to send and receive requests between itself and the processor 206. A request may be sent to the server 102 by the processor to store information for later retrieval. Further, the processor 206 may send a request to the server 102 so that the server 102 can generate a corresponding request (e.g., send a request to a 3P system, device, or process information before sending a request back to the processor 206), make logic decisions based on the received request data and other stored data, and/or store the request data. The server 102 may receive a request from the processor 206 corresponding to a request originating from the nurse call system 118 (or other system component), the request may be associated with a room, patient, device, etc. The server 102 may send data corresponding to a request originating from the nurse call system 118 (or other system components). In an embodiment, the server 102 may send data corresponding to a request originating from a device. In an embodiment, the server may generate and send a request as the request initiator. The functionality of the server is described in more detail herein.

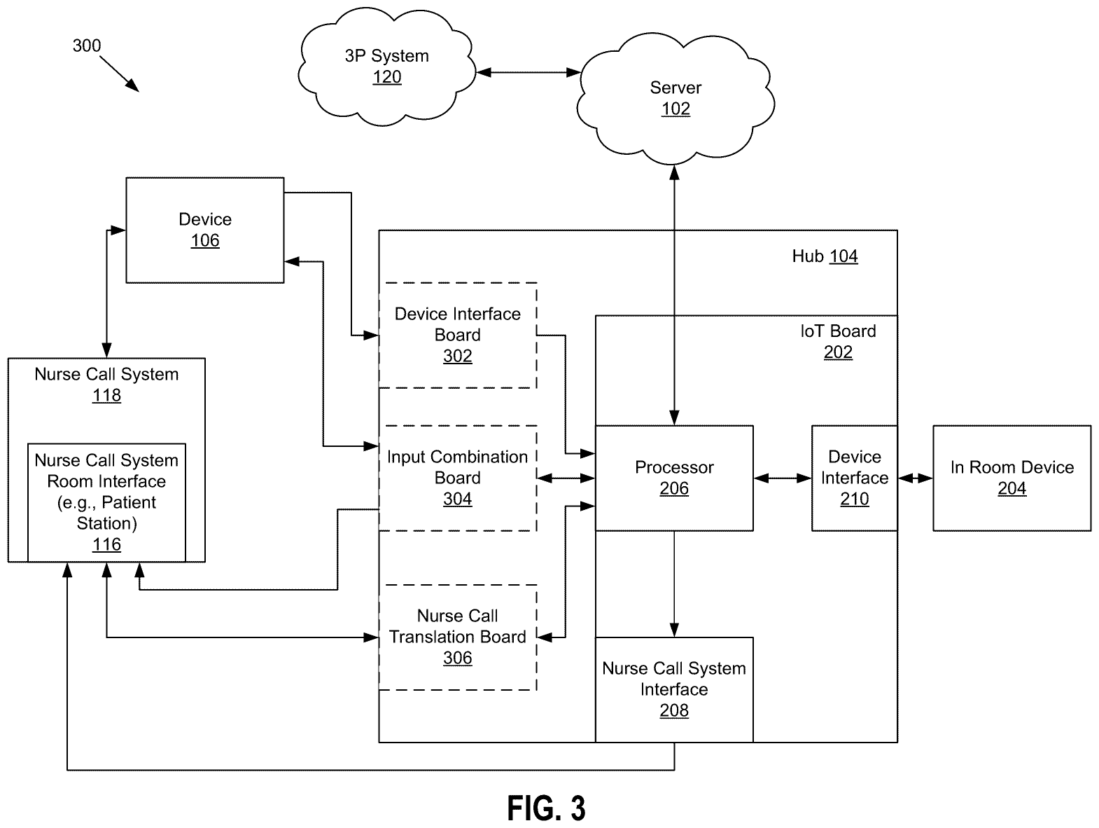

Turning now FIG. 3 which illustrates a block diagram of a system according to an embodiment. The system 300 includes a hub 104, an IoT board 202, a processor 206, an in-room device 204, a device interface 210, a nurse call system interface 208, a nurse call translation board 306, a nurse call system room interface 116, and a nurse call system 118, an input combination board 304, a device interface board 302, device(s) 106, a 3P system 120, and a server 102.

The 3P system 120 functions similarly to how it was already described in relation to system 100. The nurse call system room interface 116 and the nurse call system 118 function similarly to how they have already described in relation to systems 100 and 200. Further, the in-room device 204, device interface 210, nurse call system interface 208, and IoT board 202 also function similarly to how they have been described in relation to system 200.

In addition to the functionality of the processor 206 already described in system 200, the processor 206 may also be configured to be communicatively coupled with a device interface board 302, an input combination board 304, and/or a nurse call translation board 306. The processor 206 may receive input from a device interface board 302. Additionally, or alternatively, the processor 206 may receive input from the server 102 and/or input combination board 304. Further, the processor 206 may also output to an input combination board, and/or a nurse call translation board 306. The device interface board 302, input combination board 304, and nurse call translation board 306 are illustrated with dashed lines in system 300 to help emphasize that any combination of the boards can be used with the system. None of the board are required, all may be used, or any combination thereof.

The server 102 in example system 300 is illustrated as being situated similarly to the server 102 in systems 100 and 200. In addition to the functionality already described with relation to systems 100 and 200 may be additional configured as described. The server 102 in system 300 is communicatively coupled to a processor 206 and to an optional 3P system 120. The server 102 may send and receive data to/from the 3P system 120 and 3P system may be used to control 3P devices. Further, the processor 206 may send information to the server 102 so that the server 102 can generate a corresponding request (e.g., send a request to a 3P system 120, device(s) 106, etc.), store data, log data, respond with a request to the processor 206, etc.

In addition to the functionality of the one or more devices 106 already described in system 100, how a device 106 may be communicatively coupled with the hub 104 is specifically illustrated in system 300. Specifically, device(s) 106 may be communicatively coupled with a device interface board 302, an input combination board 304, a device interface 210, and/or a server 102. Device(s) 106 may be able to send and/or receive data to/from a device interface board 302, an input combination board 304, a server 102, and/or (via a device interface 210,) a processor 206. In an embodiment, the nurse call system 118 may be communicatively coupled with one or more devices 106 without the need to relay one or more requests through a hub 104 and/or server 102.

The device interface board 302 allows for at least one communicative data connection to a device 106. The device interface board 302 allows for some, or all, of the request data from one or more devices 106 to be passed onto the processor 206 of the IoT board 202. Subsequently, the IoT board 202 processor 206 may send corresponding request data to the server 102, input combination board 304, nurse call translation board 306, other devices 106, and/or nurse call system interface 208.

The input combination board 304 allows for at least one communicative data connection from the hub 104 to a device 106. The input combination board 304 can send and receive data between a device 106 and the processor 206 of the IoT board 202. Further, the input combination board 304 can send data to a nurse call system room interface 116. The input combination board 304 has the capability to pass device 106 input data to the nurse call system room interface 116. Additionally, the input combination board 304 has the ability combine input from device(s) 106 and the processor 206 of the IoT board 202 and send the combined input to the nurse call system room interface 116. The input combination board 304 may receive a request from the processor 206 and subsequently send a request to device(s) 106 based on the processor's request. In an example, the input combination board 304 may have received a request, containing information, from the processor 206 and a request from a device 106 television. The television information may contain the television device ID and on/off status while the processor information may contain an instruction to turn the TV on with a certain displayed message. The information from the processor 206 and television device 106 might be combined by the input combination board 304 to allow the nurse call system 118 to turn the television on and display a certain message (e.g., “help is on the way,” “do not move,” etc.) in accordance with instructions generated by either the processor 206 and/or the server 102.

The nurse call translation board 306 allows for the processor 206 to effectively interface with the nurse call system room interface 116. Based on the type/model number/serial number, etc. of the nurse call system 118, different request formats will be supported for the communicative coupling between the nurse call system room interface 116 and the nurse call translation board 306. The nurse call translation board 306 allows data being sent between the processor 206 and the nurse call system room interface 116 to be correctly formatted for the destination component to interpret the request correctly. Thus, data can flow from the processor 206 to the nurse call system room interface 116, and vice versa. The nurse call translation board 306 may be a module that is executed on the processor 206 or may contain its own processor. Further, the nurse call translation board 306 may be customized to work with a particular model of the nurse call system 118 or may contain the ability to be communicatively coupled with various nurse call systems 118 so that the nurse call translation board 306 does not have to be installed into a hub 104 based on what nurse call system room interface 116 the hub will be interfacing with. The nurse call translation board 306 may translate the request from the request received from the processor 206 into a specific request format able to be correctly interpreted by the nurse call system room interface 116, and vice versa. Thus, the nurse call translation board 306 may translate the request format based on the specific nurse call system that is being communicated with. The nurse call translation board 306 may have the ability to translate the request format to be correctly interpreted by various nurse call systems 118, or may be specifically designed to translate request formats for a particular nurse call system 118. Once any necessary request translation occurs after the nurse call translation board 306 receives a request from a processor 206 or a nurse call system room interface 116, a request is sent to the nurse call system room interface 116 or processor 206, respectively.

As an example of how system 300 may operate, consider a monitor located outside of the room (i.e., an out-of-room first device 106) sends a first connection request to the hub 104 via a device interface board 302 requesting that the monitor be communicatively connected to a second device 106. The hub 104 then sends a second connection request to the server 102, wherein the server 102 is further configured to determine the room based on the second connection request, determine the device based on the room, and establish the connection between the second device 106 and the monitor (first device 106). As a further example, the second device 106 may send a connection request directly or indirectly to the server 102 to have the server 102 establish a connection between the second device 106 and a first device 106.

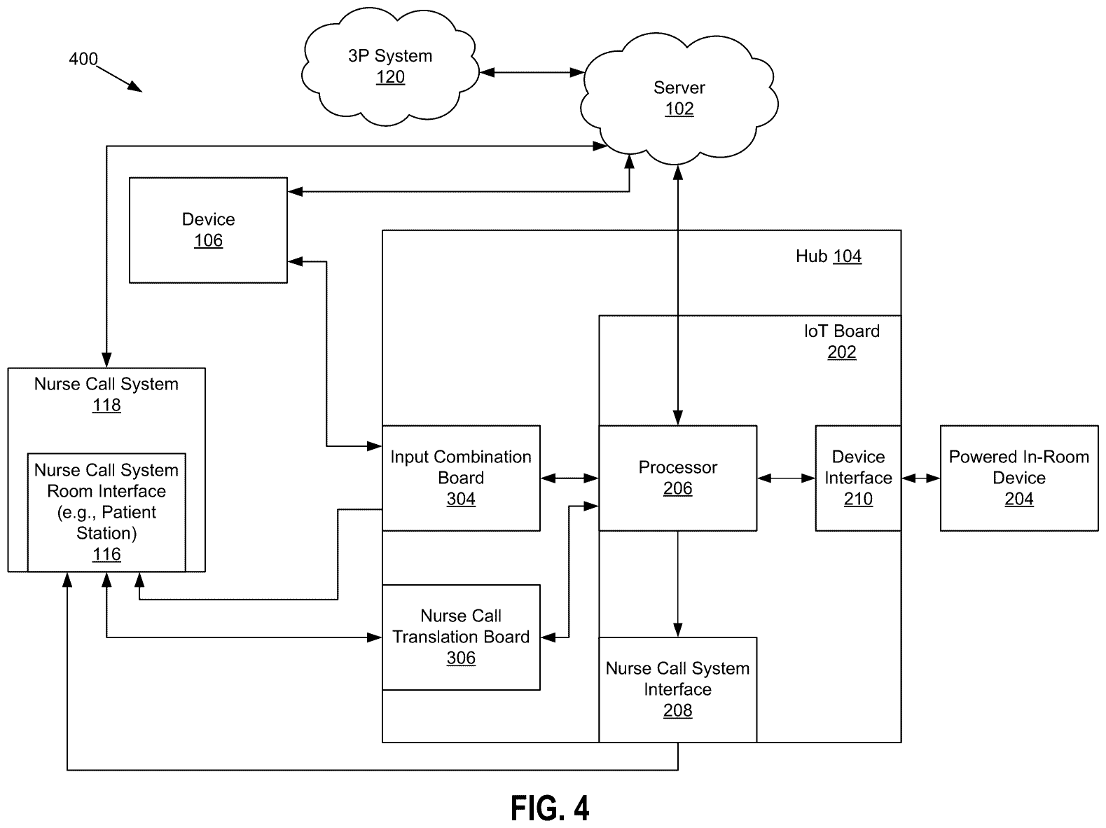

Turning now FIG. 4 which illustrates a block diagram of a system according to an embodiment. The system 400 includes a hub 104, an IoT board 202, a processor 206, a nurse call system interface 208, a nurse call translation board 306, a nurse call system room interface 116, a nurse call system 118, a 3P system 120, a device interface 210, a device 106, and a server 102. These components have already been described above and can be configured to function in the ways discussed above, or how they will be further described.

Unlike the example system 300, example system 400 shows a device 106 does not need to be connected to a 3P input module of the hub. Further, system 400 shows that the device 106 can be communicatively coupled with the server 102. Although the device 106 in system 400 is communicatively coupled to the server 102 and the input combination board 304 of the hub 104, a device 106 could be exclusively connected to either one of the server 102 or the input combination board 304. Additionally, certain devices 106 from the one or more devices represented by the device 106 system component may be communicatively coupled to the server 102 while one or more devices 106 may be communicatively coupled to the input combination board 304 and/or a device interface board (in systems in which a device interface board was included).

Lastly, system 400 also illustrates how in an example embodiment, the nurse call system 118 may be communicatively coupled to the server 102 as an alternative to, or in addition to, the hub 104 being communicatively coupled to the nurse call system via a nurse call system room interface 116.

An example of how system 400 may be used is for sending a request from a device 106 to the nurse call system 118 where the nurse call system 118 subsequently sends a request back to the device 106. A device 106 associated with a room of a hospital may send a first request to a server 102. The device 106 may be associated with a user, room, etc. The device may be located inside the room of the patient and may be operable by the patient. The server may then receive the first request with information to be sent to a nurse call system 118 of the hospital. The server 102 may then determine a permission to make the first request by determining that at least one of a user of the device 106 or the device 106 itself is permitted to make the first request. Further, the server 102 may determine, upon the requisite permission, the room based on the first request. Further still, the server 102 may determine based on the room, an identifier associated a hub 104 located in the room.

Additionally, in an embodiment, the server 102 may store, during a set-up of device 106, a first association between the room and an identifier associated with the user, the patient, and/or the device 106. The server 102 could also store, based on a set-up of the hub 104, a second association between the room and the identifier associated with the hub 104. The server could also look up associations that include the first association and the second association in response to the first request. The first association may associate a first device identifier of the device 106 with a room identifier of the room where the device 106 is located, and the second association may associate a second device identifier of the hub 104 with at least one of the room identifier or a bed identifier of a bed within the room.

The server 102 may then send to the hub 104 based on the identifier, a second request that corresponds to the first request, the second request causing the hub 104 to send the information to the nurse call system 118 via a nurse call system room interface 116 in the room. In one embodiment, the hub 104 will send the information to the nurse call system room interface 116 via the nurse call translation board 306. In another embodiment, the hub 104 will send the information to the nurse call system room interface 116 via the input combination board 304. The hub 104 (via a nurse call translation board 306, for example) may subsequently receive a fourth request from the nurse call system 118 via the nurse call system room interface 116. The server 102 may then subsequently receive from the hub 104, a third request associated with at least one of the room or the patient, wherein the third request is based on the fourth request of the nurse call system 118. The server 102 may then determine the room based on the identifier associated with the hub 104 and determine the device 106 based on the room. Then the server may send, to the device 106, a fifth request that corresponds to the third request.

As another example of system 400, consider a system 400 where the hub is powered by a dedicated power source. Further, the hub in system 400 is configured to have a device interface board 302, an input combination board 304, and a nurse call translation board 306. The hub 104 may be in a room of a hospital and configured to be connected, via a nurse call system room interface 116 in the room, with a nurse call system 118 of the hospital. Consider a scenario where a device 106 sends a request to the nurse call system 118 and the nurse call system 118 responds to the device 106 with a subsequent request.

A device 106 associated with the room may send a first request to the server 102, the first request may be associated with the room or a patient in the room. The request can be sent from the device 106 to the server 102 with or without first being routed through the hub 104. The server 102 may determine a permission to make the first request by determining that a user of the device 106 or the device 106 itself is permitted to make the first request. Once a permission is determined, the server 102 may determine the room based on the first request, and further determine an identifier associated with the hub 104 based on the room. The server 102 may then send, to the hub 104 based on the identifier, a second request that corresponds to the first request. Once the hub 104 receives the second request from the server 102, the hub 104 may generate, at the nurse call translation board 306 or the input combination board 304, based on the nurse call system 118, a third request that corresponds to the second request and that comprises the information from the first request. The input combination board or the nurse call translation board 306 of the hub 104 may then send the third request to the nurse call system 118 via the nurse call system room interface 116. In response to the third request sent to the nurse call system room interface 116, the hub 104 may then receive at the nurse call translation board 306, from the nurse call system room interface 116, a fourth request associated with the room or the patient which contains second information to be sent to the device 106. A fifth request that corresponds to the fourth request based on a format usable by the device 106, may subsequently be generated at the hub 104 processor 206. The fifth request may then be sent to the server 102. Once the server 102 receives the fifth request, the server 102 determines the room based on the identifier associated with the hub 104 and may then determine the device 106 based on the room. Once the server 102 determines what device 106 the request is intended for, the server 102 may then send, to the device 106, a sixth request that corresponds to the fifth request and that comprises the second information originating from the nurse call system 118.

As yet another example of the system's capabilities, after a nurse call system 118 receives a request corresponding to an initial request from a device 106 sent to the server 102. The hub 104 may be configured to receive, from the nurse call system 118 and via the nurse call system room interface 116, a first request associated with one of the room or the patient, second information about the first request to be sent to the device 106. The hub 104 may then generate a second request that corresponds to the first request based on a format usable by the device 106 and send the second request to device 106 via an input combination board 304 or via the server 102.

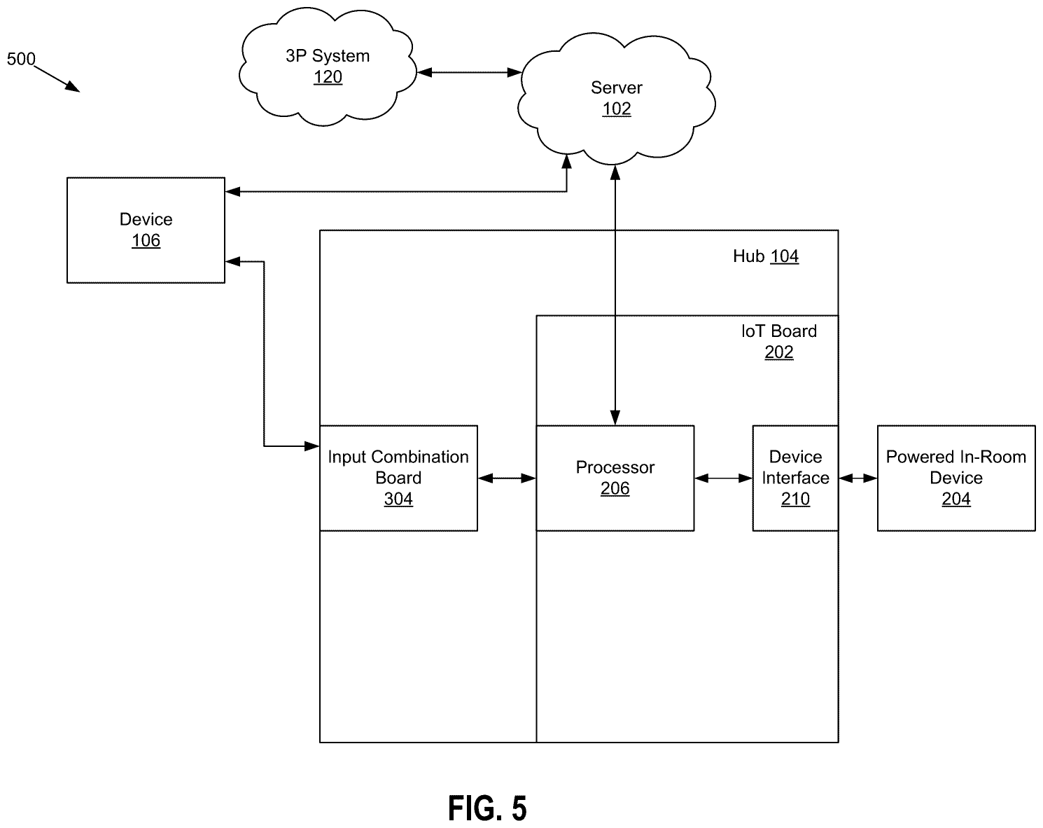

Turning now FIG. 5 which illustrates a block diagram of a system according to an embodiment. The system 500 includes a hub 104, an IoT board 202, a processor 206, a 3P system 120, a device interface 210, a device 106, and a server 102. These components have already been described above and can be configured to function in the ways discussed above, or how they will be further described.

Example system 500 shows that a hub 104 does not need to be communicatively coupled with a nurse call system. In some embodiments, the hub 104 is communicatively coupled with one or more devices 106 (e.g., Powered in-room device 204) and/or a server 102 and is not communicatively coupled to a nurse call system. In an embodiment, the hub 104 is situated like the hub 104 illustrated in system 500, and the server is communicatively coupled to one or more nurse call systems.

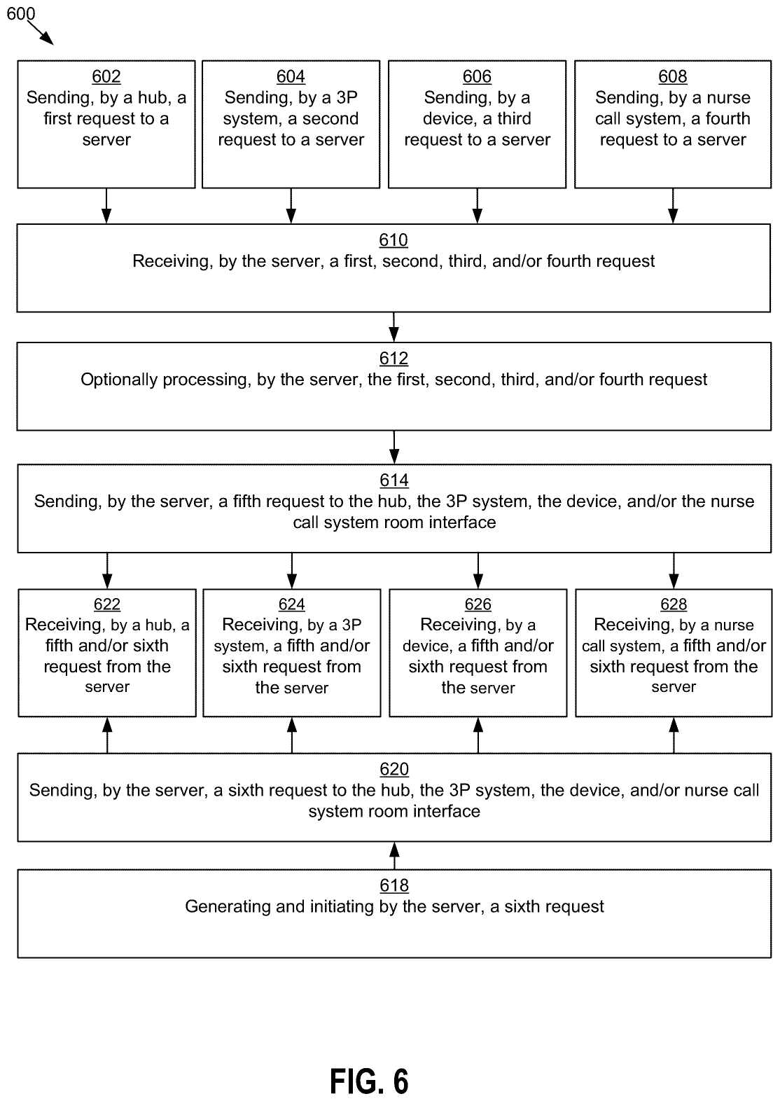

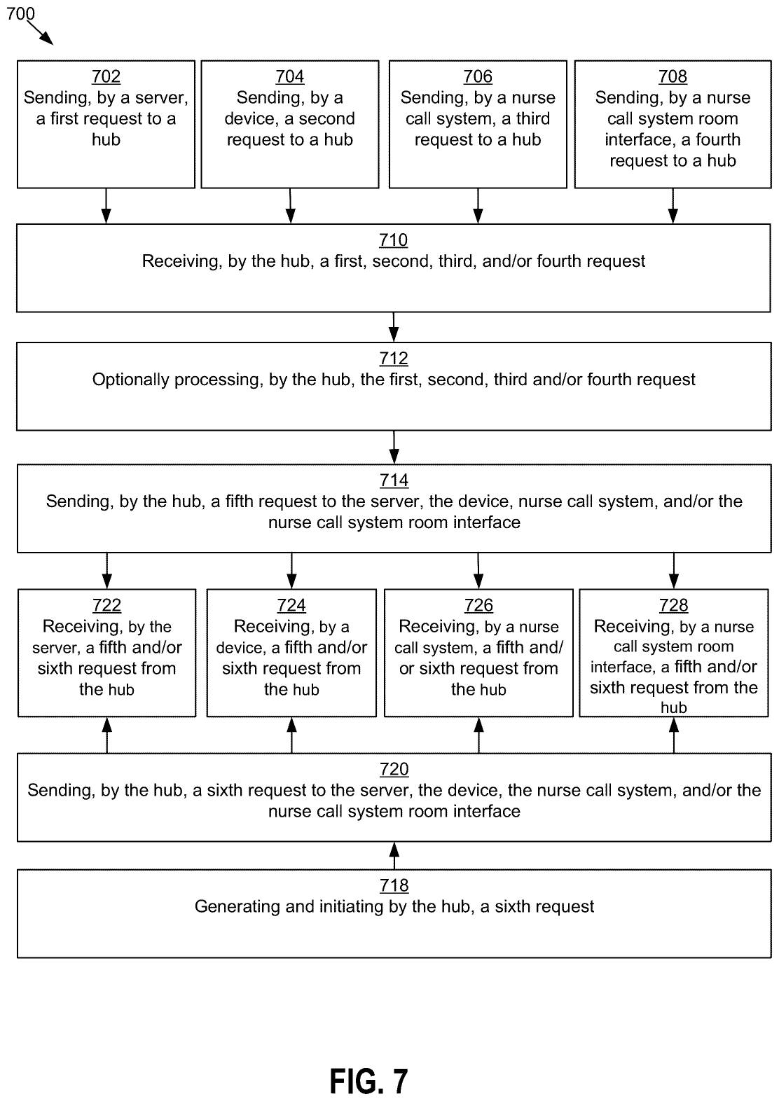

Next, the systems shown in FIGS. 1-5 can be used to perform the methods described with respect to FIGS. 6 and 7.

FIG. 6 shows another possible method embodiment of the systems disclosed herein. Example method 600 shows a flow diagram illustrating methods of sending a request to and/or from a server, in accordance with the present disclosure.

In step 602, a hub may send a first request to the server. The first request may have been initiated and generated by the hub (e.g., the hub has determined that a condition has been satisfied and sends the first request). Alternatively, the first request may correspond to, but not match a prior request received by the hub (e.g., the first request may have had the format translated or the content changed, etc.). The first request may match a request received by the hub and may merely be passed along, without modification from the input first request received. The operation of the hub has already been described in more detail above. The first request may also be the result of a prior request sent to the hub by the server or by one or more devices.

In step 604, a 3P system may send a second request to the server. The second request may be a response to a prior request received from the server, may have been initiated by the 3P system, may have been triggered by component within the 3P system, etc.

In step 606, a device (e.g., in-room device, patient-device, etc.) may send a third request to the server. The request may have been initiated by the device, the hub, the user, may be in response to a server request, may be in response to a nurse call system request, etc.

In step 608, a nurse call system may send a fourth request to the server. The request may have been initiated by the nurse call system, the hub, the user, may be in response to a server request, etc.

In step 610, the first, second, third, and/or fourth requests are received from the hub, 3P system, the device, and/or the nurse call system, respectively. Some of the things the server may do are translate the message format, alter the message content, store received request information, log information, send information to other system components, etc. The functionality of the server and what it may proceed to do after receiving a request have already been described in more detail above.

In step 612, the server may perform optional processing on the request(s) received as discussed in the methods already described.

In step 614, the server may, as a result of receiving a first, second, third, and/or fourth request, and any optional processing send a fifth request to the hub, 3P system, the device, and/or nurse call system. The hub, 3P system, device, and the nurse call system may or may not be the same hub, 3P system, device, and nurse call system from steps 602, 604, 606, and 608, respectively. In an embodiment, the server is the final destination of the first, second, third and/or fourth request and no correlating request need be sent as a result of the first, second, third request, and/or fourth request (e.g., the hub or the one or more devices sent data to the server for storage).

The server may also be capable of generating and initiating a sixth request without the need of first receiving any input from other system components. This capability is illustrated in step 618. For example, based on stored information, the server may close the shades at a certain time of day, desire to query a device based on a time interval to see if a patient is sleeping, synchronize data with the hub, etc. Many other possibilities exist for why the server would initiate and generate a fifth request for the server to send. These and related operations of the server do not require a preceding request and therefore the server is able to be the request initiator. Similar initiation of requests may be done by the hub in some embodiments.

The server may send the server generated and initiated sixth request to any of the hub, 3P system, device, and/or nurse call system in step 620 to be received by the respective components in steps 622, 624, 626, and 628.

FIG. 7 shows another possible method embodiment of the systems disclosed herein. Example method 700 shows a flow diagram illustrating methods of sending a request to and/or from a hub, in accordance with the present disclosure.

In step 702, a server may send a first request to the hub. The first request may have been initiated and generated by the server (e.g., the server has determined that a condition has been satisfied and sends the first request). Alternatively, the first request may correspond to, but not match a prior request received by the server (e.g., the first request may have had the format translated or the content changed, etc.). The first request may match a request received by the server and may merely be passed along, without modification from the input first request received. The operation of the server has already been described in more detail above. The first request may also be the result of a prior request sent to the server by the hub, a 3P system, or by one or more devices.

In step 704, a device (e.g., in-room device, patient-device, etc.) may send a second request to the hub. The request may have been initiated by the device, the server, the user, may be in response to a hub request, may be in response to a nurse call system request, may be in response to a nurse call system room interface request, etc. The request can indicate a corresponding user action taken with respect to an in-room device. For example, a user of the in-room device may have pressed a button on the device to request assistance (e.g. emergency assistance or non-emergency assistance such as for lights to be turned on/off or a drink to be brought). Further, the device may have a user interface of various forms such as an interface made up of buttons, dials, a screen, a touch screen, microphone, or any combination of these interface user input techniques, or other user input techniques. One of skill in the art would recognize other user input options that could be used. The in-room device would then communicate the user input to the hub by sending the third request to the hub in step 706.

In some scenarios, it may be beneficial for a device to only send first information in a second request to the hub and second information in a separate request to the server. In other scenarios it may be useful for a device to only send a single request to either the server or hub.

In step 706, a nurse call system may send a third request to the hub. The request may have been initiated by the nurse call system, the server, the user, may be in response to a hub request, etc.

In step 708, a nurse call system room interface may send a fourth request to the hub. The request may have been initiated by the nurse call system, the server, the user, may be in response to a hub request, etc.

In step 710, the first, second, third, and/or fourth requests are received from the server, the device, the nurse call system, and/or the nurse call system room interface, respectively. The hub may receive the request via an input combination board, device interface, or processor. Some of the things the hub may do are translate the message format, alter the message content, store received request information, log information, send information to other system components, etc. The functionality of the hub and what it may proceed to do after receiving a request have already been described in more detail above.

In step 712, the hub may perform optional processing on the request(s) received as discussed in the methods already described. The hub may transform the format of the request from the request format received into a request format to be received by a subsequent component (e.g., a nurse call translation board, an input combination board, a nurse call system interface, a server, a nurse call system room interface, a processor, etc.). For example, the processor may transform the request format into a format that can be used by the nurse call translation board to send a fourth request to the nurse call system room interface. As another example, the input combination board may receive the second request from a device via a hub processor and then generate a fourth request for outputting to a nurse call system room interface. As yet another example, the input combination board may receive the second request from a device and send a corresponding request to the processor which then changes the request format before sending a subsequent request to the nurse call translation board so that the nurse call translation board can send a fourth request to the nurse call system room interface.

The hub might also perform logic operations using the received requests before sending a communication to a subsequent component. Example logic operations might evaluate whether the second request comes from a specific in-room device ID with a particular permission level before forwarding a corresponding request to a subsequent component. The logic operations may be able to be performed with information stored locally at the hub or may require that information stored at the server be obtained before the evaluation of conditions is complete. The logic operations might also look for, as an example, whether input received by a server or hub from a different device represents a particular value at the same time a first, second, third, and/or fourth request is received. Many different logic combinations based on users, permissions, devices, timing, sequence of events, and other data capable of being collected by the system should be obvious to one of skill in the art, with the benefit of the present disclosure, to use in setting and using logic operations at the hub and/or server level.

In an embodiment, if a logic condition is satisfied, the message content is changed and/or the request format is changed. If message content is not changed (possibly due to lack of logic conditions being satisfied and requiring the message content to be altered) from the first request to the second request, then the second request has the same content as the first request. If the request format translation occurs or the message content changes between the hub receiving a request and sending a request, then the received request is not identical to the request sent by the hub due to the format change of the request and/or the message content change. On the other hand, if neither a request format translation occurs nor a message content change occurs, the request sent by the hub would be identical to the request received by the hub.

In one example, a hub may have received a second request from a device to have the nurse call system lower the window shades of the room and therefore needs to pass along the request, in the same or translated request format to the nurse call system room interface. In another example, the hub may use information previously stored on the server and/or hub to determine that a certain action should be performed by the nurse call system (e.g. the nurse call system contains information that the television is off and that a patient fall event has occurred so the server determines that the television should be turned on with text displayed to tell the patient to stay in place until help arrives) and then send a corresponding action request to the nurse call system via the nurse call system room interface (e.g., patient station). As yet another example, a request may be received from the server or a device to turn on the lights, but the hub also has stored or receives further information representing that the patient is sleeping (e.g., heart rate monitor device has sent data to the hub and/or server, using an in-room device camera), then the logic operations may result in the request being disregarded. Alternatively, the same parameters could cause a different logic condition to alter the message content (instead of disregarding the request altogether) of the request to have it represent a request to lower the room temperature to a better sleeping temperature before sending a corresponding fourth request to the nurse call system room interface or server that is connected to a 3P system. In an embodiment, a second request may be received from a device (e.g., at-home spouse device) to turn off the lights, and the hub also has stored or receives further information representing that the patient is sleeping (e.g., heart rate monitor device has sent data to the hub and/or server, using an in-room device camera), then the logic operation may result in translating the request into a fourth request with a format required by the nurse call system room interface to turn off the lights. Further, the same logic conditions may then generate and initiate an additional different fourth request to the nurse call system room interface to turn off the television. A server may also be used to perform any or all of the above-described logic operation capabilities.

In step 714, the hub may, as a result of receiving a first, second, third, and/or fourth request, and any optional processing, send a fifth request to the server, the device, the nurse call system, and/or the nurse call system room interface. The server, device, the nurse call system, and the nurse call system interface may or may not be the same server, device, nurse call system, and nurse call system room interface from steps 702, 704, 706, and 708, respectively. In an embodiment, the hub is the final destination of the first, second, third and/or fourth request and no correlating request need be sent as a result of the first, second, third request, and/or fourth request (e.g., the server or one or more devices sent data to the hub for storage).

The hub may also be capable of generating and initiating a sixth request without the need of first receiving any input from other system components. This capability is illustrated in step 718. For example, based on stored information, the hub may close the shades at a certain time of day, desire to query a device based on a time interval to see if a patient is sleeping, synchronize data with the server, etc. Many other possibilities exist for why the hub would initiate and generate a fifth request for the hub to send. These and related operations of the hub do not require a preceding request and therefore the hub is able to be the request initiator. Similar initiation of requests may be done by the server in some embodiments.

The hub may send the hub generated and initiated sixth request to any of the server, device, nurse call system, and/or nurse call system room interface in step 720 to be received by the respective components in steps 722, 724, 726, and 728.

Method 700 can be implemented by the processor, the IoT hub, the device interface, the nurse call system interface, the device interface board, the input combination board, and/or the nurse call translation board, or any combination thereof.

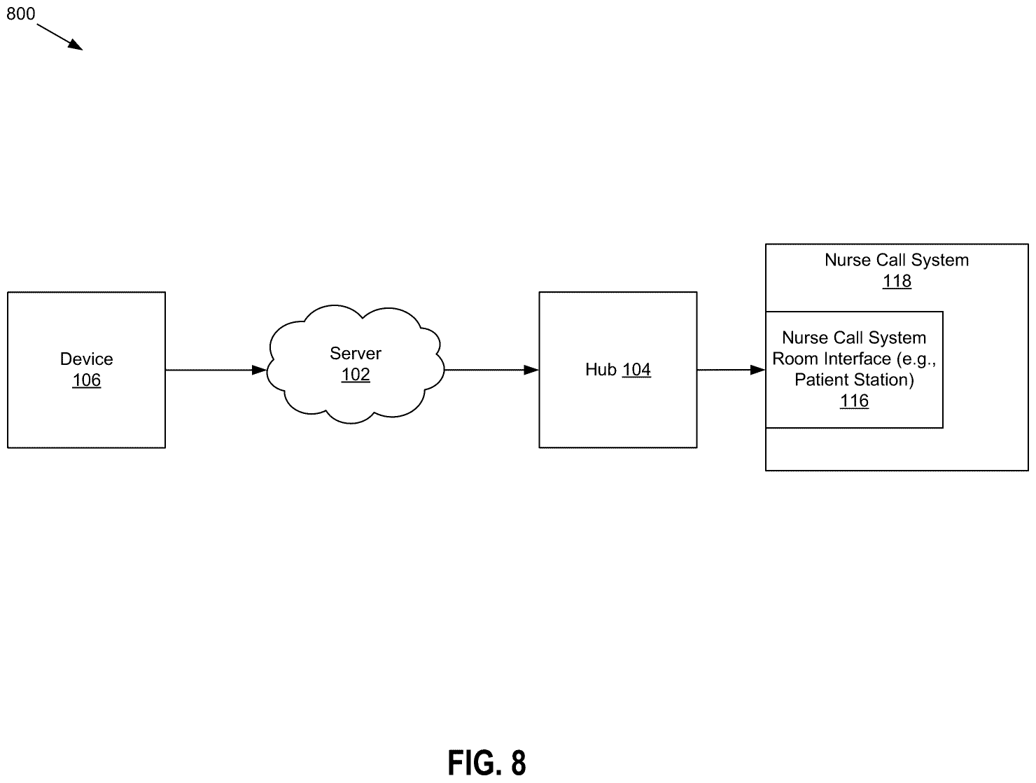

FIG. 8 illustrates another exemplary block diagram of a system 800 according to an embodiment, in accordance with the present disclosure.

System 800 is an example of how data may flow from a device 106 to a server 102, to a hub 104, to a nurse call system room interface 116, so that the nurse call system can receive a corresponding request originating from the device 106.

In an embodiment, a device 106 (e.g., phone, tablet, computer) uses a web browser to access a bring-your-own-device (BYOD) interface (e.g., a GUI of an application executing on the device 106) for communicating with a server 102, the interface to the server allowing room control, television control, clinical requests, etc. to be entered into a nurse call system room interface 116 (e.g., patient station) via a hub 104. In an embodiment, the device using the BYOD interface is only authorized to control a specific room.

In an embodiment, a device 106 is a hospital-owned device running an application (e.g., a mobile application executing on this device) to facilitate server 102 communications with the hub 104. The device may allow for a user (e.g., patient) to control the room, television, clinician requests, etc. that are entered into the nurse call system room interface (e.g., patient station). In an embodiment, the device 106 or the application running on the device 106 knows which room the user is in.

In an embodiment, members of a patient's care team (e.g., nurses, doctors, etc.) may use various devices 106 (e.g., phones, tablets, desktop computers) to control devices within a patient's room. In an embodiment, the devices 106 used by the patient's care team may control the in-room devices 106 by using a mobile application or a web portal so that room functionalities such as television control, clinical requests into the nurse call system room interface (e.g., patient station) can be controlled. In an embodiment, the patient's care team can place requests for a specific room, a specific patient, for a group of patients, for a group of rooms (e.g., turn of all room lights on the floor), etc.

A device 106 may know which room it is in based on information received by the device 106 from a user, another device 106, a hub 104, and/or a server 102, etc. For example, a user of a mobile phone device 106 may be prompted to enter in a text field which room the device 106 is within. In an example, a device 106 may recognize which room it is in based on GPS location, which router is being communicated with, which hub 104 is closest, a nearby Bluetooth beacon, system configurations on the device 106 level or server 102 level (e.g., database information associating a device to a room, a device 106 to a user, facial recognition of a user in a room paired with the user associated with the device 106), etc. In an embodiment, the device 106 does not have knowledge of the room the device 106 is in, this may mean that the device 106 does not store a room ID (or equivalent) on the device 106. For example, the device may not know which room it is in but only receives instructions from a hub 104 and/or server 102 which knows what room the device 106 is in (e.g., because the device 106 is associated with a room in a database accessible by the hub 104 and/or server 102). That is to say, that a first device 106 (e.g., an in-room lamp) may not receive instructions initiated by a second device 106 that is not associated with the room (e.g., user device in another room) because a hub 104, server 102, and/or second device 106 determined that the instructions should not be transmitted to the first device 106. Thus, devices and/or users may be associated with a particular room in memory of a device 106, server 102, and/or hub 104, etc. Further, device 106 and/or user location may be recognized on the fly through various means such as position of the device 106.

In an embodiment, a user and/or device 106 may be associated with more than one room (e.g., patient care team may have control over at least one device 106 in one or more rooms). In an embodiment, the patient care team is associated with a room but does not have requisite permissions to control one or more devices 106 within the room (e.g., patient care team may be able to turn off the lights in the room because they are associate with the room and have permissions but may not be able to turn off the television in the room because they are associated with the room but do not have permissions). How permissions may be evaluated, set, and adjusted are described in more detail herein (in the description of system 100, for example).

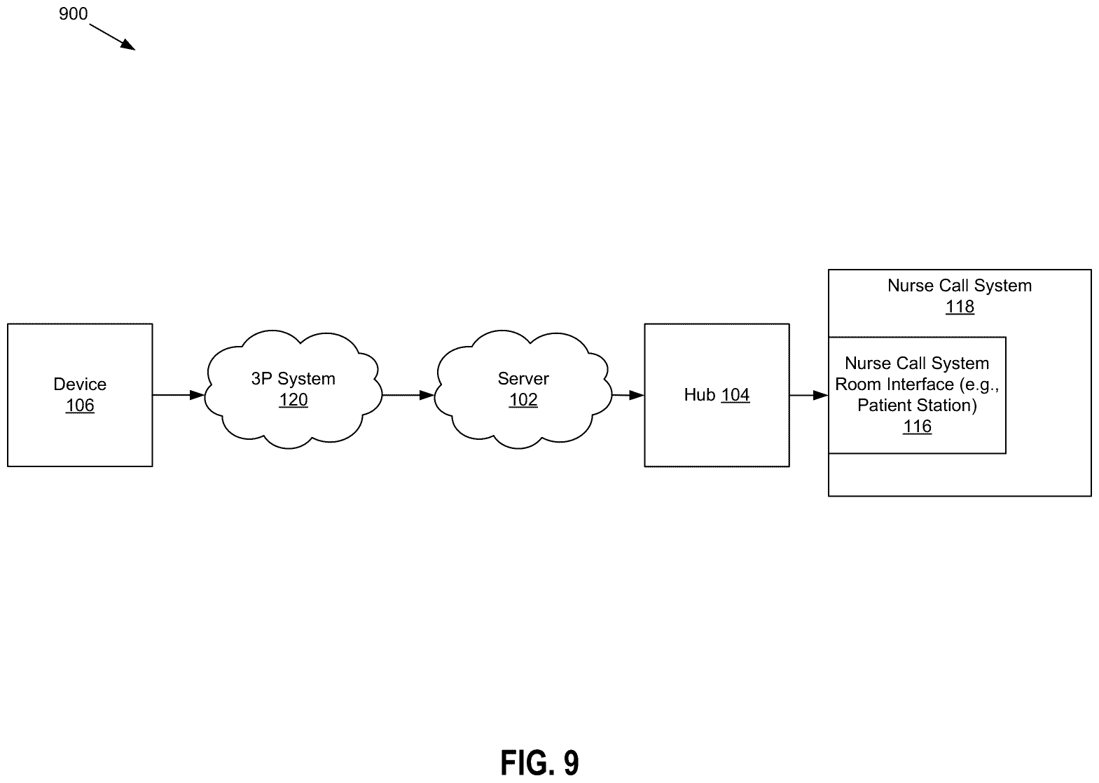

FIG. 9 illustrates another exemplary block diagram of a system 900 according to an embodiment, in accordance with the present disclosure.

System 900 is an example of how data may flow from a device 106 to a 3P system 120, to a server 102, to a hub 104, to a nurse call system room interface 116, so that the nurse call system 118 can receive a corresponding request originating from the device 106.

In an embodiment, a patient uses a hospital-owned device 106 (e.g., tablet, television) that is running an application (e.g., third party application, first party application, etc.) allowing for the control of the television, clinician requests, etc., to be placed to the nurse call system room interface 116 (e.g., patient station). The hospital-owned device 106 that is running an application may communicate with a 3P system 120, which in turn may communicate with a server 102 so that the hub 104 which is communicatively coupled to the server 102 can receive a request that causes the hub 104 to send a corresponding request to the nurse call system room interface 116. In an embodiment, the 3P system knows the assignment and location of the patient (e.g., due to the information being input into the device 106 by a patient or care team member, by communicating with the server 102, by communicating with the hub 104). In an embodiment, a 3P system server is capable of initiating a request without receiving a corresponding request from a third party device. In an embodiment, the patient does not need to interact directly with the third party device (e.g., monitor camera).

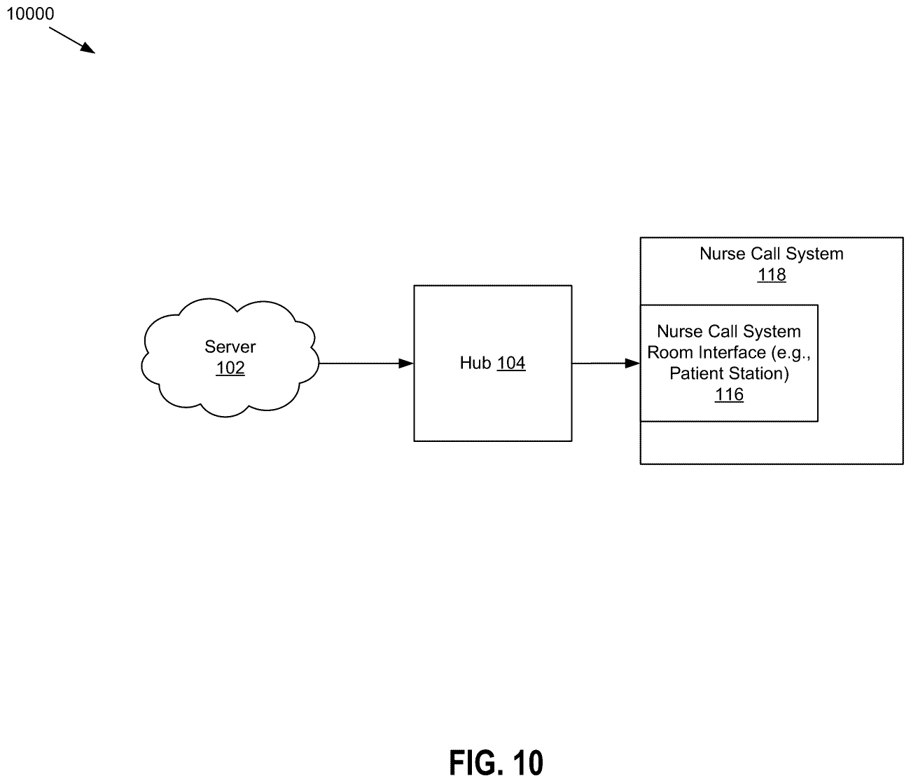

FIG. 10 illustrates another exemplary block diagram of a system 1000 according to an embodiment, in accordance with the present disclosure.

System 1000 is an example of how data may flow from a server 102, to a hub 104, to a nurse call system room interface 116, so that the nurse call system 118 can receive a corresponding request originating from the server 102.

In an embodiment, the server 102 uses logic on individual data or a plurality of data to initiate the control of a room, television, clinical request, etc. to be placed into a nurse call system room interface 116 (e.g., patient station). As an example, the server initiates the lights of a room to turn on at a certain time of day to help patients wake up. The server 102 may initiate instructions using information from one or more requests that have been saved by the server 102 or another system accessibly by the server 102 (e.g., a patient has saved in the server 102 that they want their shades opened at nine in the morning, patient has been scheduled for a meeting with the doctor at noon, so the television volume is turned down two minutes before the meeting is set to start, a server 102 receives information that a person has entered the room and it is daytime, so the lights in the room are instructed to be turned on, etc.)

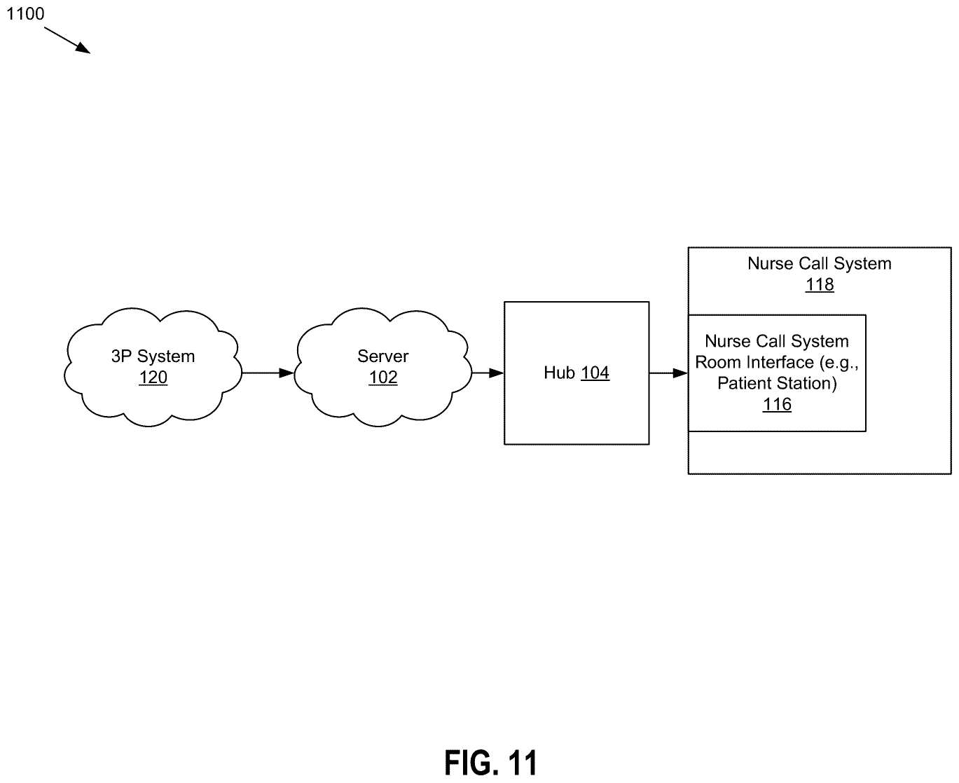

FIG. 11 illustrates another exemplary block diagram of a system 1100 according to an embodiment, in accordance with the present disclosure.

System 1100 is an example of how data may flow from a 3P system 120, to a server 102, to a hub 104, to a nurse call system room interface 116, so that the nurse call system 118 can receive a corresponding request originating from the 3P system 120.

In an embodiment, the 3P system 120 uses logic on individual data or a plurality of data to initiate the control of a room, television, clinical request, etc. to be placed into a nurse call system room interface 116 (e.g., patient station). In an embodiment, the 3P system is the nurse call system 118. As an example, a code blue button may be pushed on a patient station or nurse station which causes all of the lights to turn on in the corresponding room (the corresponding room may be known based on information saved in memory on an in-room device and/or saved on the hub 104, server 102, 3P system 120, etc.).

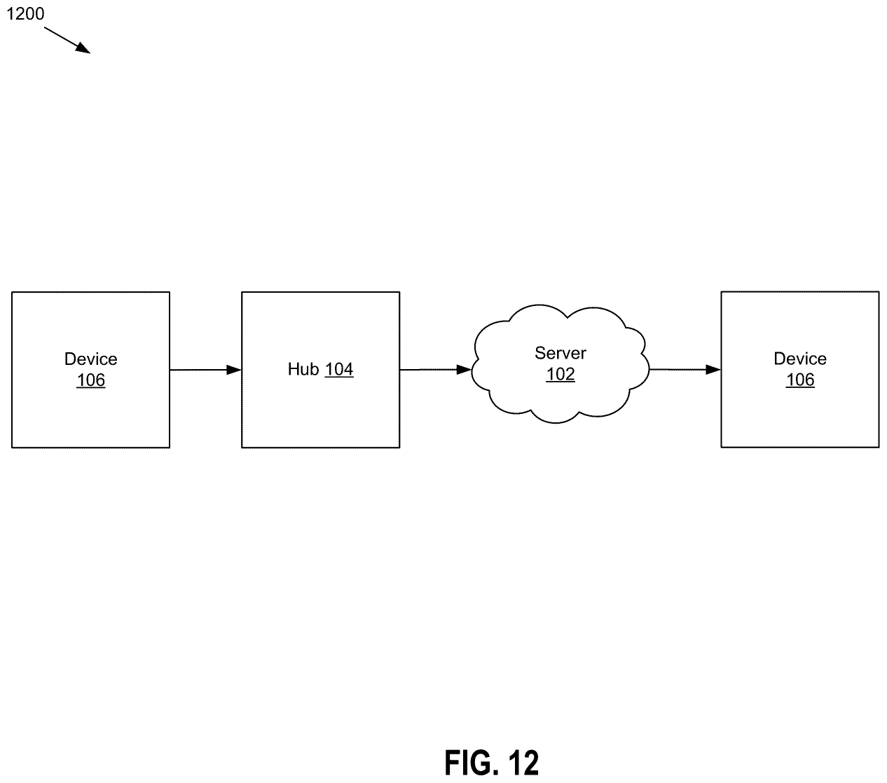

FIG. 12 illustrates another exemplary block diagram of a system 1200 according to an embodiment, in accordance with the present disclosure.

System 1200 is an example of how data may flow from a first device 106, to a hub 104, to a server 102, and then to the same or a different second device 106.

In an embodiment, a first non-server connected medical device 106 (e.g., IV pump) associated with a patient or healthcare location sends data to the hub 104, the hub 104 then sends a request to a server 102, causing the server 102 to then send a request to a second device 106 which causes the different second device 106 (e.g., patient device, clinician device, etc.) to display insight on the care that has, is, and/or will take place. In an embodiment, the server 102 additionally or alternatively sends a request to a 3P system as a result of a request received from the hub 104. In an embodiment, a device 106 that is the destination for requests from a hub 104, server 102, device, or 3P system is determined based on a device 106 ID, a room associated with the device 106, a user associated with the device 106, etc. Various combinations of information collected and stored by the systems described herein are capable of being used to determine where collected requests should be sent and/or stored.

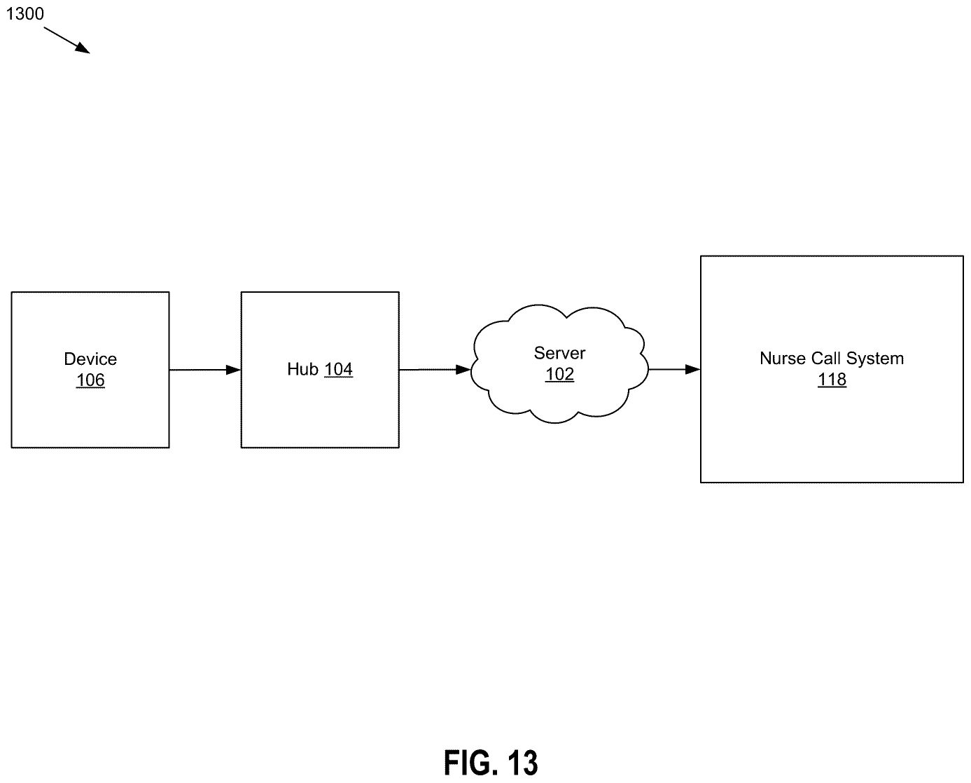

FIG. 13 illustrates another exemplary block diagram of a system 1300 according to an embodiment, in accordance with the present disclosure.

System 1300 is an example of how data may flow from a device 106, to a hub 104, to a server 102, to a nurse call system 118 as a result of the request originating from the device 106.

In an embodiment, a non-server connected medical device 106 (e.g., IV pump) associated with a patient or healthcare location sends data to the hub 104, the hub 104 then sends a request to a server 102, causing the server 102 to then send a request to a nurse call system 118. In an embodiment the hub evaluates logic to determine if conditions are met to trigger a request related to room control, television control, clinical request, to be placed into the nurse call system 118. Conditions have already been described, but may depend on a room ID, patient ID, device ID, data received by a device, hub, and/or server. Conditions may depend on where requests are received from or could be sent to. Many parameters that conditions may rely on would be obvious to one having ordinary skill in the art in light of the present disclosure. In an embodiment, the server 102 is additionally or alternatively used to evaluate conditional logic for determining if conditions are met to trigger a request related to room control, television control, clinical request, to be placed into the nurse call system 118. In an embodiment, a nurse call system room interface 116 (e.g., patient station) is used to receive a request from the server and send a corresponding request to the nurse call system 118.

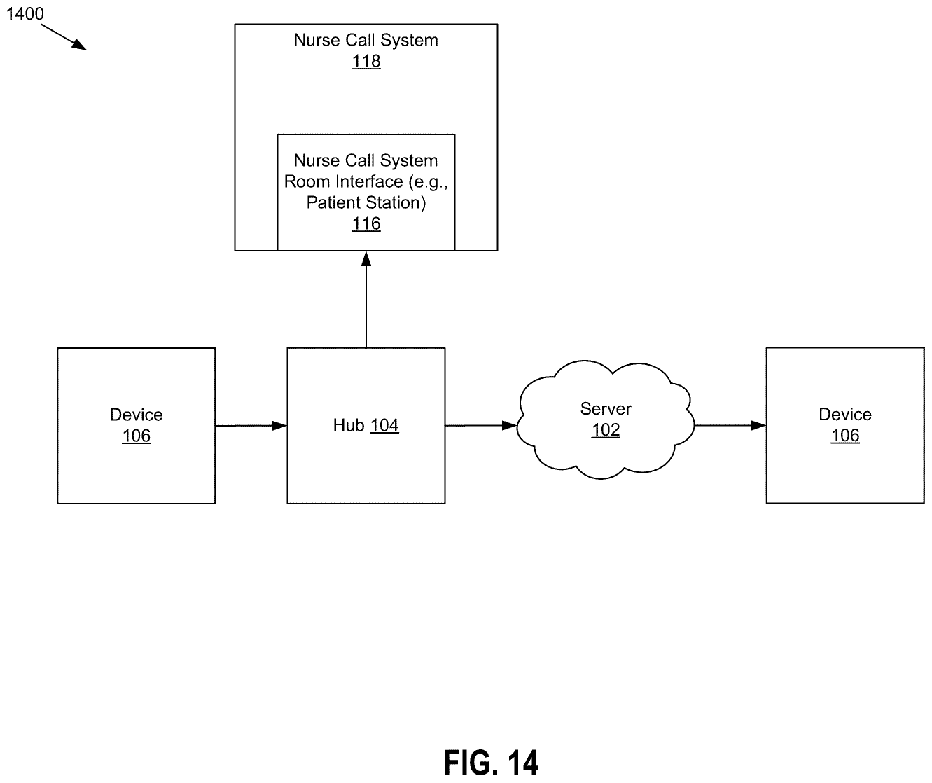

FIG. 14 illustrates another exemplary block diagram of a system 1400 according to an embodiment, in accordance with the present disclosure.

System 1400 is an example of how data may flow from a first device 106, to a hub 104, to a server 102, and then to a same or different second device 106 as a result of the original request sent by the first device 106. Additionally, or alternatively, as a result of receiving a request from the first device 106, the hub 104 may send a request to a nurse call system room interface 116 (e.g., patient station), the nurse call system room interface then sends a corresponding request to the nurse call system 118.

In an embodiment, the first device 106 is a non-server connected device associated with a patient or a healthcare location (e.g., hospital bed device 106). The first non-server connected device 106 sends a request to the hub 104. As a result of the request, in an embodiment, the hub 104 may send a corresponding request to the nurse call system room interface 116 (e.g., patient station). In an embodiment, the hub 104 uses conditional logic using information within the request (e.g., bed height information, bed power status, patient occupancy condition of a bed or a room based on a camera device, etc.) received by the hub 104 before sending a subsequent request. In an embodiment, the hub 104, as an addition or alternative to the request sent to the nurse call system room interface 116, sends a request to the server 102, causing the server 102 to then send a request to a second device 106 that is the same or different than the first device 106. In an embodiment, the second device 106 is running an application (e.g., web application, mobile application, etc.) and displays information about the patient's care. In an embodiment, the server 102, additionally, or alternatively sends a request to a 3P system as a result of receiving the request from the hub 104. In an embodiment, the server uses conditional logic using information within the request received by the server 102 before sending a subsequent request.

Depending on requests sent by a device 106, the request may be initially destined to a certain system (e.g., device 106, hub 104, server 102, 3P system) without knowing where the information contained within the request will be used. For example, a bed device 106 may update it's plugged in status with a server 102. This information may be sent to the server based on routine data collection for later analysis. In an embodiment, the information obtained by the server 102 from the bed device 106 may trigger a condition to be satisfied at the server level that causes an alert to be generated by the server 102 and sent to the nurse call system 118. Thus, a request from a first device can be sent to the intended second device, server, and/or 3P system recipient, where the intended recipient may then relay or generate additional requests using the information obtained from the request from the first device.

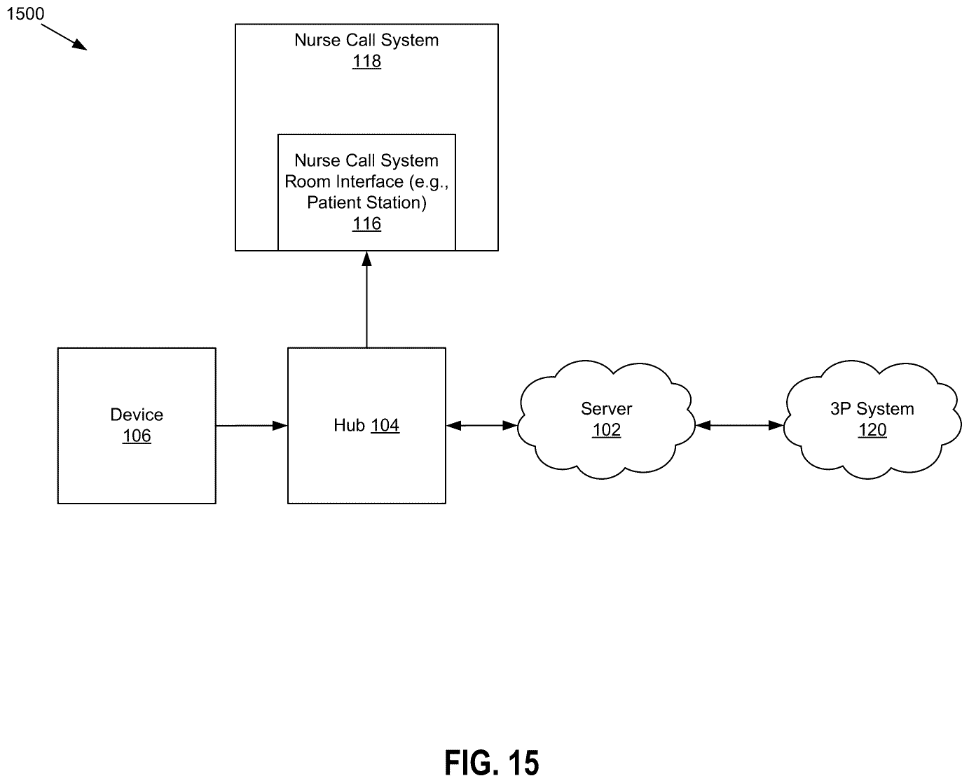

FIG. 15 illustrates another exemplary block diagram of a system 1500 according to an embodiment, in accordance with the present disclosure.

System 1500 is an example of how data may flow from a device 106, to a hub 104, to a server 102, to a 3P system 120, back to the server 102, back to the hub 104, and then to a nurse call system room interface 116 (e.g., patient station) for communication with the nurse call system 118.

In an embodiment, a non-server connected medical device 106 (e.g., hospital bed) is associated with a patient or healthcare location. The non-server connected medical device 106 sends a request to the hub 104, the hub 104 then sends a request to a server where logic is used by the server 102 to analyze data received within the received requests and/or other data obtained by or stored on the server 102. In an embodiment, the server 102 sends a request to the 3P system 120 to obtain more information for evaluation of conditional logic (e.g., bed rails are down and the patient (emergency medical record) EMR data from the 3P server indicates the patient is a high fall risk). If after, receiving the requests from the hub 104 and/or 3P system 120, the server 102 determines a condition is satisfied, in an embodiment, the server 102 sends a request to the hub 104, causing the hub 104 to sends a request to a nurse call system room interface 116 (e.g., patient station) allowing for the room, television, clinical request, etc. to be controlled (e.g., place an emergency call). In an embodiment, the hub 104 sends a request to one or more devices 106 after receiving the request from the server 102. In an embodiment, the hub 104 additionally, or alternatively performs conditional logic. In an embodiment, upon conditional logic being satisfied, the server 102 sends a request to the nurse call system 118.

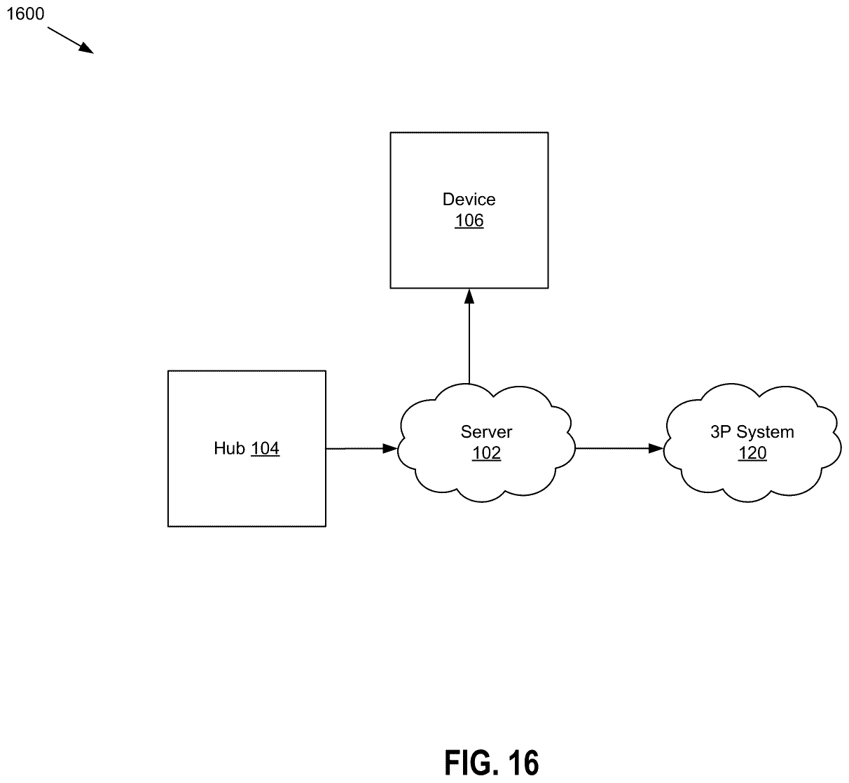

FIG. 16 illustrates another exemplary block diagram of a system 1600 according to an embodiment, in accordance with the present disclosure.

System 1600 is an example of how data may flow from a hub 104, to a server 102, which in turn, sends a request to a 3P system 120 and/or a device 106.

In an embodiment, the hub may act as a sensor or beacon and is associated with a patient or healthcare location (e.g., used as a real-time locating system). Using the hub in such a way can allow for the hub to detect the presence of a caregiver or object (e.g., hospital bed) and send requests to the server and/or 3P system causing events to be performed such as completing open patient requests, turning off alarms, causing a device to store and/or display certain information, etc. Using the hub is such a way can also allow for voice recognition and/or patient tracking.

In an example, a bed device 106 may not be plugged into an outlet. The bed device 106 may send a signal to a server 102 and/or a hub 104 that indicates the bed device is not plugged in. The hub 104 and/or server 102 may determine of logic conditions have been satisfied (e.g., bed device 106 has not been plugged in for 2 minutes, bed device 106 is not plugged in and a patient is on the bed device 106, bed device 106 is not plugged in and a patient is assigned to the room, bed device 106 is not plugged in and a nurse is not in the room, etc.) before activating an alarm (e.g., nurse call system alert, sound alarm in the room, flashing lights on a device 106, etc.). Further, the logic conditions for evaluating whether an action should occur based on a bed being plugged in or not plugged in may further rely on an IR sensor in the room, embedded within a device 106, embedded within the hub 104, a camera device 106, a camera embedded within a device 106, a camera embedded within the hub 104, a current sensor embedded within a bed device 106, a current sending device attached to the power chord of the bed device 106, etc.

In an embodiment, the proximity of a device (e.g., bed) to a hub may be determined by a camera IR sensor, and/or sonar sensor embedded within the hub. The hub may use such information to determine if a bed is in the room and not plugged in versus a bed not being in the room and not being plugged in, for example.

In an embodiment, similar conditions can be used to determine when a state of a device was changed (e.g., when a bed was plugged in or unplugged). Further, an embodiment also allows for the determination of whether a bed was never plugged in once it was brought into the room or whether it was once plugged in after being brought into the room. Such determinations could be useful in determining the context of a situation within the room, if a bed should be plugged in, and/or evaluate further logic conditions.

Many combinations exist as to what devices and data could be used from each device, in order to trigger an alarm condition, or any other condition capable of being evaluated by the hub 104, server 102, or a device 106.

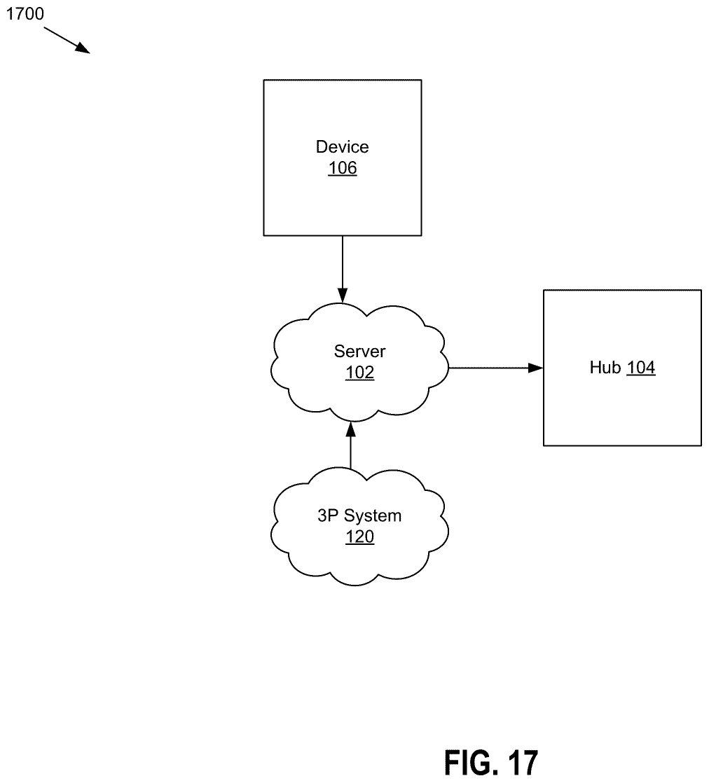

FIG. 17 illustrates another exemplary block diagram of a system 1700 according to an embodiment, in accordance with the present disclosure.

System 1700 is an example of how requests may flow from a device 106 and/or a 3P system, to a server 102, resulting in the server 102 sending a request to a hub 104.

In an embodiment, the hub may emit light (e.g., pulsing lights, projection onto the room ceiling, etc.), scent (e.g., scents meant to cause a soothing reaction), and/or sound (e.g., siren, beeps, real-time voice output, etc.). The hub may receive a request to do so from the server based on logic performed at the server and/or requests received by the server from one or more devices 106 and/or one or more 3P systems. In an embodiment, the hub may emit sound, lights, scents, etc. as a result of logic performed by the hub.

FIGS. 8-17 are examples of how data may flow and be used in embodiments enabled by the disclosure. FIGS. 8-17 are not meant to be limiting, many other possible combinations and use cases exist using the system components and their capabilities that are described herein.

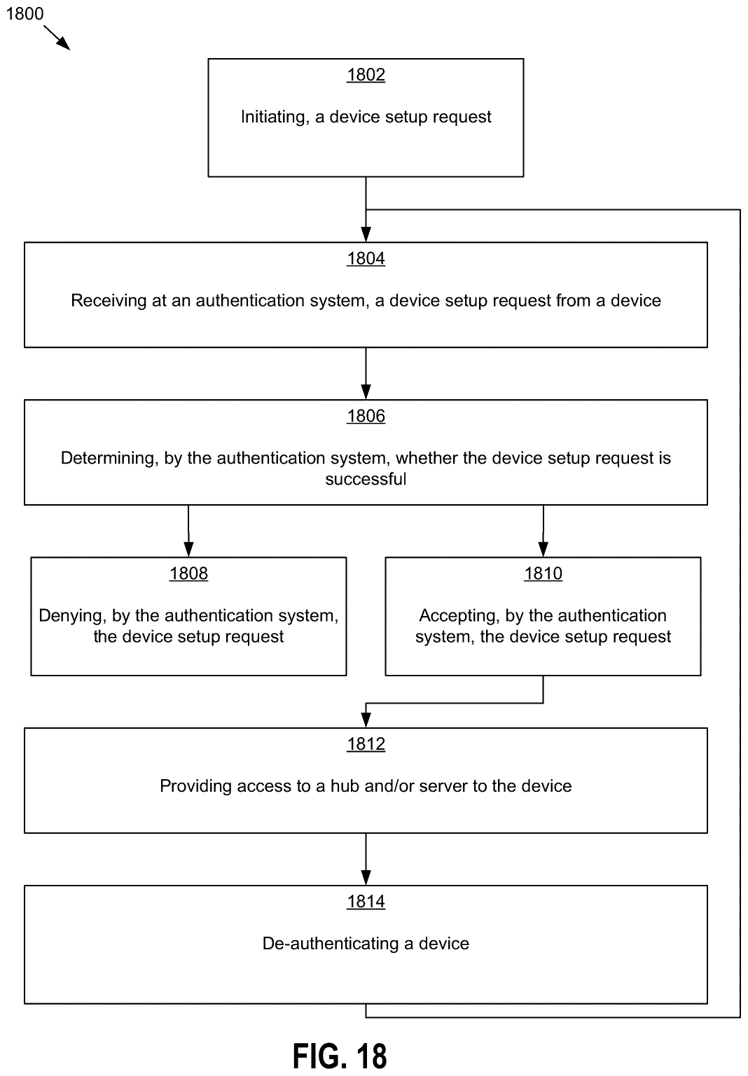

FIG. 18 shows a flow diagram illustrating methods of authenticating a user and/or device for communication with a hub, server, and/or third party system, in accordance with the present disclosure.

step 1802, a device setup request may be initiated. A device setup request may be initiated by a user in an embodiment. In an example, a device setup request is initiated by the user taking a picture of a QR code (e.g., the QR code encodes an IP address), taking a picture of the QR code and then clicking on the QR code, visiting a website, clicking a link, connecting to a particular router network, and/or connecting to a particular network, etc. In an example, a QR code is displayed to a user. The QR code may be displayed on a television screen in a room, may be on a bracelet of a patient, may be on a paper placed in the room, may be affixed to a bed in a room, and/or may be on a screen of a hub, etc. In an embodiment, a device setup request is initiated by the device. For example, the device may try to re-authenticate to an authentication system that has authenticated the device before without any user input (e.g., a user checks into a hospital and is assigned a room for a second time, one month after their initial visit, the device connects to the Wi-Fi of the hospital and the authentication system then determines whether the device and/or user should be authenticated).

At step 1804, an authentication system may receive a device setup request from a device of the user. The authentication system may be a third party system (3P system). The authentication system may grant access to data retained by a 3P system, server, and/or hub. Further, the authentication system may be implemented by the hub and/or server. Thus, in an example, the device which sent the device setup request is wirelessly communicatively coupled with the hub and the hub is capable of authenticating a device and/or user of the device, or the hub is connected to a different device that is capable of doing so. Similarly, the server may carry out such functions, in an embodiment.

At step, 1806, the authentication system determines whether the device setup request is successful. The determination may be made based on the router that the device is connected to, the network that the device is connected to, the location of the device, the IP address of the device, the user of the device (e.g., indicated by an email, identification number, username, etc.), a password (or other credentials) associated with a user, a MAC address, and/or based on electronic medical records. Further, any combination of the listed determination factors may be used, in an embodiment. One of ordinary skill in the art with the benefit of this disclosure would recognize other possible means of authenticating a user and/or device for accessing the systems disclosed herein. In an embodiment, the user is an authorized user but the device is not authorized and therefore the authentication system rejects the device setup request.

At step 1808, if the authentication system determines that a device and/or user should not be granted access to a hub, server, and/or third party system, the authentication system denies the device setup request which prevents the device from obtaining access to information on the hub, server, and/or third party system.

At step 1810, if the authentication system determines that the device setup request is successful, the authentication system will accept the device setup request. In an embodiment, more than one device and/or users may be provided access by the authentication system. In an embodiment, a device and/or user is provided access to the hub, server, and/or 3P system by the authentication system but the user's or device's permission is limited by permissions and/or configurations of the hub, server, and/or 3P system.

As illustrated in step 1812, if the authentication system accepts the device setup request, the device is provided access to a hub, server, and/or third party system. For example, a user may have scanned a QR code in the room using their phone and as a result may have been prompted to enter a username and password, if the authentication system accepts the device setup request, then in response to the acceptance, the device may present information on the user interface of the device that allows a user to control devices and/or room configurations (e.g., bed, television, shades, timers, preferences, nurse calls, etc.) within the room. Therefore, the user and/or device may obtain the ability to read and/or write data from/to the server, hub, and/or a third party system.

At step 1814, a device and/or user may be deauthenticated. Such deauthentication may cause the user and/or device to lose the ability to read and/or write data to the server, hub, and/or a third party system. Deauthentication may occur based on any factor that may be used in step 1806 when determining whether a user should be authenticated. Additionally, in an embodiment, deauthentication may occur because a period of time has passed, because permissions have been changed, another device has been connected, etc.

After a device and/or user has been deauthenticated, it may be possible for the user and/or device to reauthenticate soon after deauthentication and/or after a set period of time has passed.

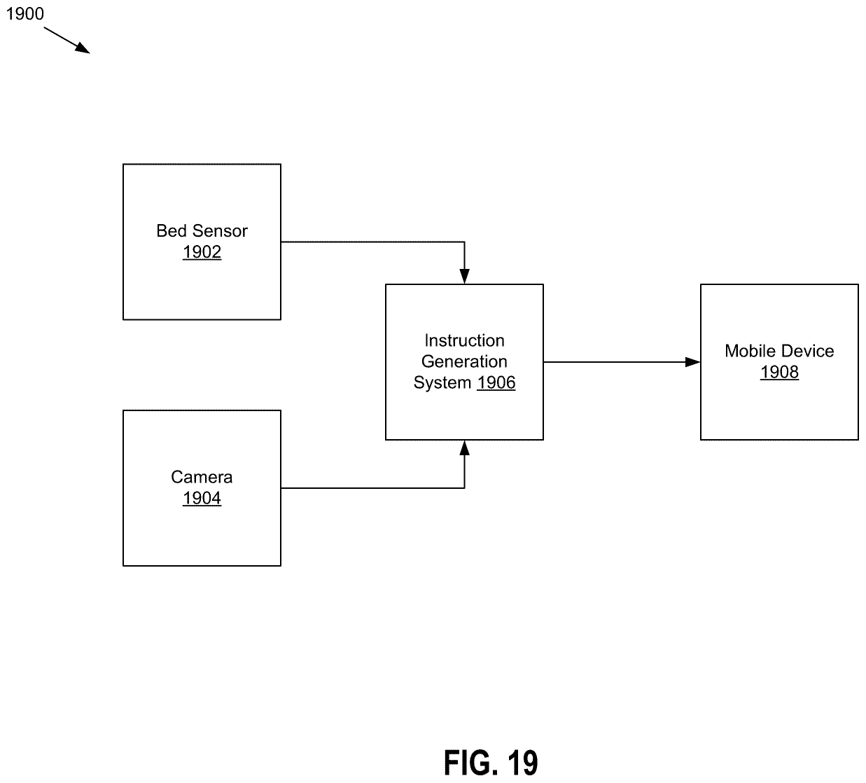

FIG. 19 illustrates another exemplary block diagram of a system 1900 according to an embodiment, in accordance with the present disclosure. The system 1900 is illustrated to include a bed sensor 1902, a camera 1904, an instruction generation system 1906, and a mobile device 1908. System 1900 can be used to obtain first input from a first device (e.g., bed sensor 1902) and a second device (e.g., camera 1904), use the inputs as input to the instruction generation system 1906 to generate an instruction. The instruction may be transmitted from the instruction generation system 1906 to a third device (e.g., mobile device 1908). The instruction may cause the third device to perform a type of healthcare related service. For example, mobile device 1908 may present a notification (e.g., to a nurse or other healthcare professional) that indicates a patient has fallen, a patient is ready for transport, a patient should be helped, a doctor is needed, a bed or other device has not been plugged in or has been unplugged, a patient needs medical attention, etc.

Although system 1900 depicts a bed sensor 1902 as the first device, a camera 1904 as the second device, and a mobile device 1908 as the third device, the specific types of devices have been shown for simplicity of explanation, and are intended to be exemplary, not limiting. Other devices are also considered. For example, any of the first device, the second device, and/or the third device may be any combination of the bed sensor 1902 or other sensor (e.g., a light sensor, a temperature sensor, a motion sensor, an accelerometer, a gyroscope, a pressure sensor, a proximity sensor, a flow sensor, a magnetic field sensor, a humidity sensor, a sound sensor, a force sensor, a chemical sensor), a bed, a light, an alarm, a mobile device (e.g., a tablet, a mobile phone, a smart watch, etc.), a user device, a camera, a speaker, a hub (e.g., hub 104 as described herein), a 3P system (e.g., 3P system as described herein), device 106 as described herein, a nurse call system (e.g., nurse call system 118 described herein), a patient station (e.g., described above), a pillowcase (e.g., pillowcase 110 described herein), a bed controller (e.g., bed controller 108 described herein), a hall monitor (e.g., hall monitor 112 described herein), a PED (e.g., PED 114 described herein), an electronic medical records (EMR) storage device, a smart outlet, a television, a display, a thermostat, a patient monitoring device (e.g., a heart rate sensor, a respiratory sensor, an oxygen saturation sensor), a magnetic resonance imaging (MRI) machine, an X-ray machine, an ultrasound device, a blood analyzer, an infusion pump, an electrocardiograman electroencephalograph (EEG), a glucose monitor, a wearable device (e.g., a smartwatch), a mass detection device, a ventilator, a defibrillator, a pacemaker, a capnograph, or sensor used in a healthcare facility and/or for a type of healthcare related service. The first device, the second device, the third device, and/or any other devices of system 1900 may include an interface. The interface may include a graphical user interface (GUI). The interface may include a communication interface enabling communication via Wi-Fi, Bluetooth, wirelessly, a wired connection, etc. The interface may include a user interface may include a button, a dial, a touchscreen, a camera, a speaker, a light, a display, and/or a sensor, etc.

Additionally, more or less devices (e.g., more or less than two devices) may be used to transmit input to the instruction generation system 1906. Further, instructions generated by the instruction generation system 1906 may be transmitted to more than one device. In certain embodiments, instructions are sent to multiple devices that have the same type (e.g., both devices are a user device, both devices are an MRI machine), multiple devices that perform the same type of healthcare related service (e.g., a smart watch that monitors a heartbeat and an ECG), multiple devices that have the same type and perform the same type of healthcare related service (e.g., an first ECG and a second ECG), devices of different types, and/or devices that perform different types of healthcare related services.

Further, each of the devices that can be included in the system 1900 (e.g., devices generating input for the instruction generation system 1906 and/or receiving instructions from the instruction generation system 1906 may provide a type of healthcare related service. A type of healthcare related service may be a service that relates to assessment, prevention, and/or treatment in an healthcare facility. For example, the type of healthcare related service may include emergency care, preventative care, rehabilitative care, long term care, hospital care, diagnostic care, primary care, home care, mental health care, dental care, prenatal care, substance abuse treatment, nutritional support, etc.

Further details of how system 1900 can be used are described below along with other embodiment configurations.

In the illustrated example, the bed sensor 1902 may be capable of sensing whether a patient is in a bed, how much the patient weights, how the patient is positioned in the bed, and/or whether a patient is moving in the bed, to the bed, or from the bed. The bed sensor may be physically coupled with a bed (e.g., the bed includes the bed sensor 1902). First input received from the bed sensor 1902 may be transmitted to the instruction generation system 1906. The first input may be represented by bed sensor data generated by the bed sensor 1902 (e.g., values (e.g., representing pressure/weight) at positions). The first input may be represented by processed bed sensor data (e.g., indicating a patient is sleeping, indicating a patient is leaving the bed) generated by processing the bed sensor data.

In the illustrated example, the camera 1904 may be capable of sensing whether a patient is in a bed, how the patient is positioned in the bed, whether a patient is moving in the bed, to the bed, or from the bed, whether someone else is in a room, who else is in the room, if light are on in the room, etc. Second input received from the camera 1904 may be transmitted to the instruction generation system 1906. The second input may be represented by camera data (e.g., pixel values) generated by the camera 1904. The second input may be represented by processed camera data (e.g., data indicating how many people are in the room) generated by processing the camera data.

The first input and the second input may be transmitted to the instruction generation system 1906. The instruction generation system 1906 may be configured to receive input from the bed sensor 1902, the camera 1904, and/or one or more other devices to generate an instruction. The instruction generation system 1906 may process received input (e.g., the first input and the second input) to generate the instruction. The instruction generation system 1906 may process the input using a rule based approach such as evaluating if a set of conditions are met and then generating an instruction based on which conditions are met. The instruction generation system 1906 may process the input using one more artificial intelligence models (e.g., a classification model, a generative model). The instruction generation system 1906 may determine which device to transmit an instruction to and/or an instruction to transmit based on received input and/or a configuration of the instruction generation system 1906.

In certain embodiments, the first input and the second input are input to the AI model to generate an instruction. The AI model may generate a classification which may indicate (e.g., map to) an instruction to be transmitted to the mobile device 1908 (e.g., the third device). In certain embodiments, the classification acts as an instruction and can be transmitted to the mobile device 1908. In certain embodiments, the first input and the second input are input to the AI model to generate data (e.g., EMR data) that can be included in the instruction.

The instruction generation system 1906 may be local or remote to the bed sensor 1902, the camera 1904, and/or the mobile device 1908. For example, the instruction generation system 1906 may be physically connected to or included in one or more of the devices, may be located within inches or feet to one or more of the devices, may be located in the same room as one or more of the devices, a hub (e.g., hub 104) may include the instruction generation system 1906, may be located in the same healthcare facility as one or more of the devices, and/or may be located outside of the healthcare facility of one or more of the devices.

The instruction may be transmitted to the mobile device 1908 and/or another device. The instruction may cause and/or be used by the mobile device 1908 to perform a first type of healthcare related service. The first type of healthcare related service may be a same or different type of healthcare related service as provided by the bed sensor 1902, camera 1904, or other device that can be used with the system 1900 to receive input to transmit to the instruction generation system 1906 and/or receive instructions from the instruction generation system 1906.

In certain embodiments, the first type of healthcare related service can be a fall prevention service. For example, the mobile device 1908 may present a notification when the instruction generation system 1906 generates an instruction for the mobile device 1908 to present the notification. The instruction may be generated based on the AI model generating a bed exit classification based on the first input and the second input. The fall prevention service may generate notifications (e.g., to be presented visually, audibly, vibrationally, etc.) when a patient attempts to leave the bed, which can help prevent falls (e.g., for elderly or mobility-impaired patients).

In certain embodiments, the first type of healthcare related service can be a pressure ulcer prevention service. For example, the mobile device 1908 may present a notification when the instruction generation system 1906 generates an instruction for the mobile device 1908 to present the notification. The notification may indicate whether the patient has been determined to be at risk of developing a pressure ulcer. The instruction may be generated based on the AI model generating a pressure ulcer risk score by analyzing the patient's movement and position in bed as indicated by the first input and the second input. In certain embodiments, the instruction may indicate that the patient should be repositioned. The instruction may be transmitted to the mobile device 1908 of a nurse and/or the mobile device of the patient. The instruction can be transmitted to ensure that the patient is regularly repositioned, reducing the risk of developing pressure ulcers (bedsores).

In certain embodiments, the first type of healthcare related service can be a vital signs monitoring service. For example, the instruction generation system 1906 can be configured to generate the instruction indicating vital signs such as heart rate, respiratory rate, and body movements, and/or classifications of the vital signs (e.g., potential health issues, respiratory distress determination, cardiac problem determination). The instruction may be transmitted to the mobile device 1908 to assist in monitoring of the vital signs such as heart rate, respiratory rate, and body movements. This data can be used for early detection of potential health issues, such as respiratory distress or cardiac problems.

In certain embodiments, the first type of healthcare related service can be a sleep quality assessment service. For example, the instructions can indicate sleep patterns, including duration and quality of sleep thereby providing data to the mobile device 1908 for diagnosing and managing sleep disorders. In certain embodiments, the instruction generation system 1906 generated a diagnosis for a sleep disorder and transmits instruction to the mobile device 1908 that instruct the mobile device 1908 to present the diagnosis.

In certain embodiments, the first type of healthcare related service can be a breathing and heart rate monitoring service. For example, continuous monitoring of breathing and heart rate can help detect abnormalities early, providing timely intervention for conditions such as sleep apnea or cardiac arrhythmias. The user device may present abnormalities detected by the instruction generation system 1906 that caused the instruction generation system 1906 to transmit an instruction to present a notification of the abnormality to the mobile device 1908.

In certain embodiments, the first type of healthcare related service can be a patient presence detection service. For example, a combination of the bed sensor 1902 and the camera 1904 can detect whether a patient is present in the bed, which can be useful for ensuring patient safety and for automating certain care processes, such as turning off lights or adjusting room temperature. Accordingly, an instruction may be transmitted to the mobile device 1908 that indicates a user interface should be presented for turning lights on or off, the instruction may cause the mobile device 1908 to present a notification. Additionally, or alternatively, second instructions may be transmitted to one or more other devices, such as lights or a temperature control system. The second instructions may cause the lights to turn on or off based on the presence of a patient or other people. Other factors may also be considered by the instruction generation system 1906 for generating the instruction. For example, the instruction generation system 1906 may use a time of day as a factor (e.g., if presence detected during the night and patient is sleeping (e.g., based on sensor measurements indicating patient movement in bed), lights remain off). The instruction generation system 1906 may further consider EMRs or input from other devices when generating the instruction. In an example, the EMRs may indicate a medicine regimen for the patient and movement of the patient, the medicine regimen, and time of day may be factored into the instruction generation by the instruction generation system 1906.

In certain embodiments, the first type of healthcare related service can be an activity tracking service. For example, monitoring patient movements can provide insights into their physical activity levels, helping to tailor rehabilitation programs or assess recovery progress.

In certain embodiments, the first type of healthcare related service can be a remote monitoring service. The bed sensor 1902 can transmit data to another system (e.g., mobile device 1908, a hub, a server, the instruction generation system 1906, etc.), allowing healthcare providers, family members, and others to keep track of a patient's conditions in real-time, even when they are not physically present.

In certain embodiments, the bed sensor 1902 and/or the camera 1904 is used to provide one or more of the above described types of healthcare related services. As an example, even if the camera 1904 can provide a patient presence detection service, data transmitted by the camera 1904 to the instruction generation system 1906 could be further processed by the instruction generation system 1906. Additionally, the instruction generation system 1906 may further receive data from one or more other devices (e.g., the bed sensor 1902) and use the inputs from the camera 1904, bed sensor 1902, and/or other devices to generate the instruction to transmit to the mobile device 1908 or another device. The device the instruction is transmitted to may also provide the same service as the camera 1904, but the precision and/or accuracy of the service performance may be increased based on the processing performed by the instruction generation system 1906 in generating the instructions and/or based on the aggregation of data.

A type of healthcare related services may be provided by one or more devices, alone or in combination. For example, one or more devices may provide a type of healthcare related service of monitoring vital signs, cardiac activity, glucose, physical activity, sleep, heart rate, and/or blood content. In certain embodiments, a device may provide a type of healthcare related service of visualizing structures (e.g., an X-ray). In certain embodiments, a device may provide a type of healthcare related service of removing materials (e.g., excess fluid) from blood (e.g., in patients with kidney failure). Other services and devices to provide the service(s) would be obvious to one of skill in the art with the benefit of the present disclosure.

In certain embodiments, a same device may transmit input to the instruction generation system 1906 and receive an instruction output from the instruction generation system 1906. For example, mobile device 1908 of a patient may provide input, such as gate (e.g., walking gate) information, to the instruction generation system 1906. The instruction generation system 1906 may then generate an instruction based on the provided input (e.g., the gate), and possibly input from other devices, and transmit the instruction to the mobile device 1908. The instruction may include information about the gate of the patient, such as that the patient is walking with a limp that indicates an injury or that the patient may fall. The instruction may cause the mobile device 1908 to present a notification to the patient asking them to sit down or ask someone for help in walking.

Some example embodiments are described below. They are intended to be exemplary and non-limiting. The examples describe how different devices may be used to provide input to the instruction generation system 1906 and receive instructions from the instruction generation system 1906. The instruction generation system 1906 may generate various instructions to control devices (e.g., cause devices to store data, cause devices to present information, cause devices to be configured, cause devices to obtain data, etc.) and may transmit instructions the devices.

In certain embodiments, the instruction generation system 1906 may receive an EMR as first input from an EMR storage device. The EMR may include fall risk data for a patient. The instruction generation system 1906 may further receive second input from camera 1904, third input from a bed sensor 1902, and/or fourth input from a device indicating whether a patient is moving and being assisted by a nurse (e.g., a “sitter alarm”). The instruction generation system 1906 may use the first input, the second input, the third input, and/or the fourth input to generate an instruction. The instruction generation system 1906 may generate a determination of whether a patient is exiting a bed. In certain embodiments, if the instruction generation system 1906 determines the patient is exiting the bed, the instruction may be transmitted to a device that presents a bed exit alarm (e.g., mobile device 1908, a hall monitor, a nurse call system, etc.). In certain embodiments, the instruction is transmitted to a device in the room and causes the device to present instructions to the patient exiting the bed (e.g., instructions sent to a television in the room or a television control system and causing the television to present a message to wait for nurse assistance).

In certain embodiments, instructions can cause a television or other device to present content, messages (e.g., emergency messages), and/or educational information responsive to receiving an instruction from the instruction generation system 1906.

In certain embodiments, when a device presents a bed exit alarm, an indication of the alarm is transmitted to the instruction generation system 1906. The instruction generation system 1906 may generate an instruction to transmit to an EMR storage device. The instruction may indicate that a fall risk score should be updated for the patient. In certain embodiments, when the instruction generation system 1906 determines a bed exit is occurring or has occurred, an indication of the bed exit alarm is transmitted to the EMR storage device.

In certain embodiments, devices may obtain data that indicates when a bed is connected or not connected to power and/or a nurse call system. For example, the bed may sense the connection, the outlet or nurse call system may sense the connection, and/or a sensor may determine whether the bed is in the room and camera 1904 may determine whether the bed is connected. The obtained data may be transmitted to the instruction generation system 1906 and used to determine a bed connection score. The bed connection score may be included in an instruction generated by the instruction generation system 1906 and transmitted to an EMR storage device. In certain embodiments, the obtained data is input to the instruction generation system 1906 and used to determine if the bed is connected to power and/or the nurse call system. The determination may be included in an instruction and the instruction may be transmitted to a device such as a nurse call system or a hall monitor. The instruction may cause an indication to be presented that indicated whether the bed is connected. The indication may be useful to show the bed should be connected and it is not currently connected. Similar to the bed connection, connections of other devices can also be determined and instructions can be generated based on the determined connection. For example, a connection of a speaker may be determined.

In certain embodiments, first EMR from an EMR storage device can indicate patient information such as when a patient checks in or out of a room. Second EMR from the EMR storage device can indicate when a second patient check into the room. The instruction generation system 1906 may be configured to process the first EMR and the second EMR to determine that the change in patients to a room has occurred. The instruction generation system 1906 may determine that software of devices in the room should be updated and/or memory of devices located in the room should be updated based on the change in EMR information. For example, accounts logged into device in the room may be logged out of upon the device receiving the instruction from the instruction generation system 1906.

In certain embodiments, a device that can sense a patient presence (e.g., camera 1904, bed sensor 1902, etc.) and/or location of a patient received from a patient mobile device or patient bed may indicate that the patient is not in a room. The instruction generation system 1906 may determine that a set of nurse call alarms can be turned off and/or EMR data be updated based on the patient location and lack of presence in the room. The instruction generation system 1906 may transmit instructions to the nurse call system and/or an EMR storage device instructing that the nurse call be turned off and/or the EMR records be updated, respectively.

In certain embodiments, a speaker in a room receives an instruction from the instruction generation system 1906 after the instruction generation system 1906 determines a sounds should be presented by the speaker. The speaker, sound, and/or time to present the sound may be included in the instruction based on inputs received by the instruction generation system 1906. For example, the instructions generation may receive input indicating the a patient is anxious (e.g., is crying or pacing) and the instruction generation system 1906 may cause, via the instruction, a sound (e.g., a soothing sound, an instruction) to be presented by the system.

In certain embodiments, if a staff member is detected in a room (e.g., by a badge reader device, a hall monitor device, camera 1904, etc.), two factor authentication to devices in the room may be disabled or enabled. By disabling two factor authentication when a staff member is detected in the room, the staff member can effectively be preauthorized based on authenticating upon entering the room using the badge reader. Disabling the authentication can reduce resources used to authenticate the staff member after they have entered the room since they are already authenticated. A camera or other device near the room may detect when the staff member leaves and transmit the detected information to the instruction generation system 1906 which may determine that an instruction should be generated to cause the two factor authentication to be re-enabled. The instruction may be transmitted to one or more devices in the room (e.g., a tablet that includes patient EMR).

In certain embodiments, the instruction generation system 1906 may transmit an instruction to a bed to cause the bed to adjust position (e.g., bend, lower, etc.). For example, an instruction to lower the bed may be transmitted from the instruction generation system 1906 based on the instruction generation system 1906 receiving input from the EMR indicating a patient height, room assignment, and/or that a patient is approaching the room.

In certain embodiments, messages and/or survey answers from a mobile device (e.g., of a patient) may be input to the instruction generation system 1906. The instruction generation system 1906 may further receive nurse call indications from a nurse call system. The instruction generation system 1906 may use the received input data to determine a patient score or patient experience score. The patient score or patient risk score may be included in an instruction to transmit to an EMR storage device and cause the EMR storage device to store the patient score or patient risk score.

In certain embodiments, the instruction generation system 1906 may generate an instruction and transmit the instruction to the mobile device 1908 or another device. The mobile device 1908 or the other device may then use the instruction to generate other data that can then be transmitted to the instruction generation system 1906 and/or another instruction generation system 1906.

In certain embodiments, the instruction may indicate detection of a mass, detection of a shadow, portions of a display or other device that are illuminated, who is in a room, family of a patient is in the room, an identifier of a clinician (e.g., clinician in the room, a clinician to be sent to a room), patient privacy status (e.g., VIP, toileting), a patient is in a lift (e.g., a ceiling lift, a floor lift), equipment located in a space (e.g., a room), an identifier of a technician or other person in the room, an identifier of a vendor in room, a QR code for display, a facial expression (e.g., a facial expression of someone near the first device or second device), a body position (e.g., a body position of a person near the first device or the second device), whether a food tray is in an area (e.g., a room), whether a urinal is in use, functioning, and/or full, how long someone or something has been in an area (e.g., a room), whether a device is out of reach of a person (e.g., patient) in a space, whether a door is open or closed, which door(s) is open or closed, whether someone has washed their hands, whether someone is washing their hands, when someone washed their hands, how long someone washed their hands for, a time of day, a date, when someone was turned over in bed (e.g., with assistance and/or without assistance), when a patient checked into a room, when a patient checked out of a room, when a room has been turned over, an orientation of a device (e.g., a bed within the room, an angle of the bed), whether medication has been administered, which medication has been administered, whether disposables (e.g., gloves, needles, cups, etc.) have been used, which disposables have been used, when disposables have been used, whether privacy curtains are open or closed, which (e.g., using a curtain identifier) privacy curtains are open or closed, a hand signal made by someone, a head signal (e.g., nod) made by someone, a face signal (e.g., a frown) made by someone, when housekeeping is in a room, when housekeeping is done with a room, nurse rounding (e.g., when a nurse has visited a patient, how often, a frequency, an identifier of the nurse, etc.), language spoken in a room, a translation of language spoken in a room, an indication of voice assistance use, staff member updates via voice, and/or a condition of room.

In certain embodiments, the instruction generation system 1906 may be trained on a per patient basis.

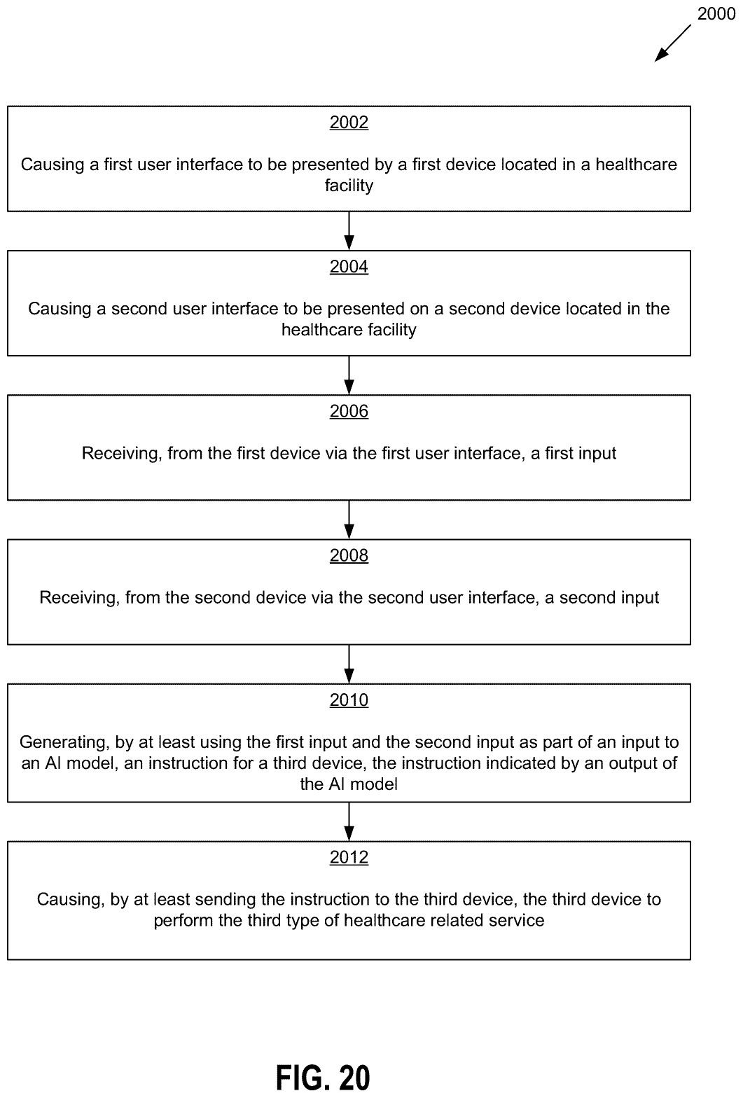

FIG. 20 shows a flow diagram illustrating a method 2000 of using an Artificial Intelligence (AI) model to receive inputs from devices to generate an instruction for another device, in accordance with the present disclosure. The method may be performed using a system such as system 1900 described above. An instruction generation system (e.g., instruction generation system 1906 described above) may perform portions of the method. The instruction generation system may include an AI model. The AI model may include a classification model. The AI model may include a generative AI model.

At step S2002, a first user interface may be caused to be presented. The first interface may be configured to enable data to be sent and/or received. The first interface may be a sensor and/or a user interface. The first interface may include a graphical user interface. In an example, the first interface may be presented by a device (e.g., a mobile device). The first interface may be presented by a first device (e.g., bed sensor 1902, a nurse call device, another device described herein). The first device may be located in a healthcare facility. The first device may be configured to provide a first type of healthcare related service (e.g., a type of healthcare related service as described above).

At step S2004, a second user interface may be caused to be presented. The second user interface may be a user interface similar to the first user interface. The second user interface may be presented by a second device (e.g., camera 1904, a EMR storage device (e.g., including a fall risk indication for a patient and associated with an identifier of the patient), another device described herein). The second device may be located in the healthcare facility. The second device may be configured to provide a second type of healthcare related service. The second type of healthcare related service may be different than the first type of healthcare related service. The second device may be a different type of device than the first type of device.

At step S2006, a first input may be received. The first input may be received from the first device. The first input may have been received via the first user interface or generated using the first user interface. The first input may may be related to the first type of healthcare related service. The first input may include at least any of the inputs described above. As an example, the first input may include a medical record (e.g., including in EMR) and/or a message. As another example, the first input may include a message received by the first device from another device located in a healthcare facility (e.g., a survey response, a chat message).

At step S2008, a second input may be received. The second input may be received from the second device. The second input may have been received via the second user interface or generated using the second user interface. The second input may may be related to the second type of healthcare related service.

At step S2010, an instruction may be generated. The instruction may be generated by the instruction generation system, which may include the AI model. The instruction may be generated by using the first input, the second input, and/or another input (e.g., from another device). The instruction may be generated to transmit to a third device (e.g., an alarm device, an EMR storage device). The third device may be configured to provide a third type of healthcare related service (e.g., a movement monitoring service, an EMR management service). The third type of healthcare related service may be different than the first type of healthcare related service and the second type of healthcare related service. The instruction may be indicated (e.g., included in) an output from the instruction generation system and/or the AI model. In an example, the third device may be instructed to present a light, sound, and/or other indication that indicates movement (e.g., of a patient, of a patient in a bed, etc.). The instruction may have been generated by the AI model based on the first input and/or the second input.

In certain embodiments, the AI model is a generative AI model and the generative AI model receives the first input, the second input, and a prompt, to generate the instruction. The prompt may indicate a desired output, examples of desired output, an example instruction, etc. The generative model may be used to generate images (e.g., for display in a room, for display by a television in a room, for display on a display outside the room (e.g., in a hall)). The generative model may be used to generate responses to questions asked. The questions may be asked using microphones or other devices. The questions may be included in the first input, the second input, and/or the prompt. The generative model may be used to generate output to be transmitted to another device. For example, the generative model may generate EMR based on the first input and/or the second input. The generative model may be configured to generate audio. The audio may be music. For example, calming music may be generated in the evening based on time input received as the first input and second input indicating lights are off in a room. The audio may be transmitted to a device located in the room.

In certain embodiments, the instruction includes a classification and/or is generated based on the classification generated by the AI model configured to generate classifications. The AI model may receive a first set of one or more inputs (e.g., from one or more devices, user interfaces) and generate a first classification based on the first set of inputs. The first classification may determine what first instruction to be generated. The first classification may indicate information (e.g., included in the first instruction) to be transmitted to a device. The first classification may indicate which device to send the instruction to.

In certain embodiments, the AI model may receive a second set of one or more inputs (e.g., from one or more devices, user interfaces) and generate a second classification based on the second set of inputs. The second classification may determine what second instruction to be generated. The second classification may indicate information (e.g., included in the second instruction) to be transmitted to a second device. The second classification may indicate which device to send the instruction to. The second device may be the same device or a different device than the device the first instruction is transmitted to. The devices may each perform healthcare related services and/or a type of healthcare related service.

At step S2012, the instruction may be transmitted to the third device. The instruction may cause (e.g., via a suggestion, instruction, prompt) the third device to perform the third type of healthcare related service.

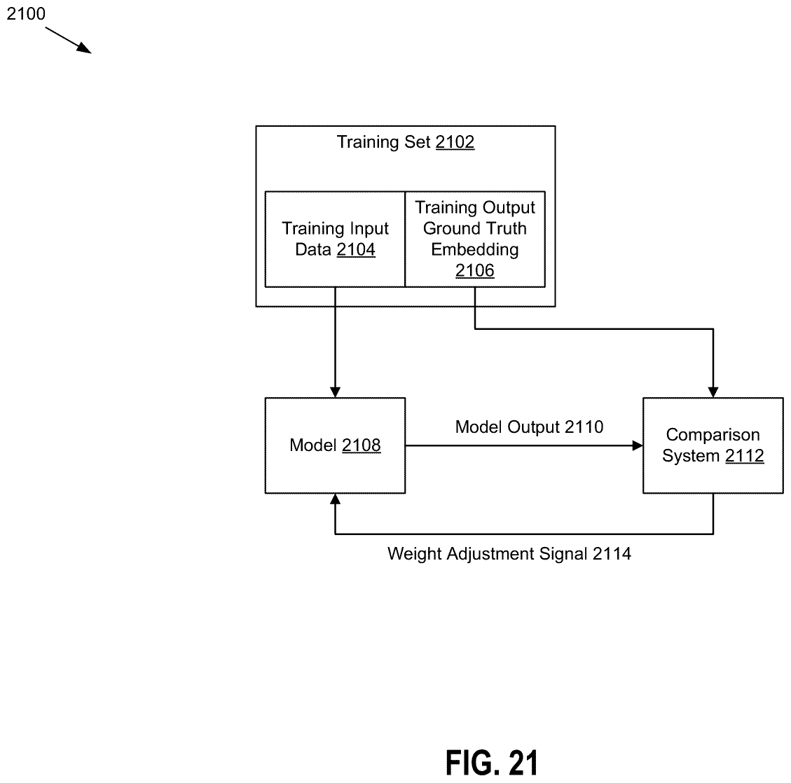

FIG. 21 illustrates an exemplary block diagram of a system 2100 for training AI models according to an embodiment, in accordance with the present disclosure. The system 2100 may include a training set 2102. The training set 2102 may include training input data 2104. The training set 2102 may include a training output ground truth embedding 2106. System 2100 may include a model 2108. The model 2108 may include a classification model (e.g., a binary classification model, a non-binary classification model, a bed exit classification model, a fall risk classification model, a bed unplugged classification model, a pressure ulcer classification model, a respiratory distress classification model, a cardiac problem classification model, a sleep classification model, heart rate abnormality classification model, a patient presence classification model, and/or a nurse presence classification model, etc.) or a generative model (e.g., a large language model). System 2100 may include a comparison system 2112.

The model 2108 may be trained using the comparison system 2112. Comparison system 2112 may compare model output 2110 (e.g., a text encoding, a classification encoding (e.g., an embedding), an encoded instruction) generated by the model 2108 to a training output ground truth embedding 2106 (e.g., a text encoding, a classification encoding, an instruction encoding). Training output ground truth embedding 2106 may be associated with the training input data 2104 used by the model 2108 to generate the model output 2110. Based on the comparison of the training output ground truth embedding 2106 and the model output 2110, the comparison system 2112 may generate a weight adjustment signal 2114. The weight adjustment signal 2114 may be transmitted to the model 2108 to cause adjustment of weights for the model 2108. Through iterations, the adjustments can train the model 2108 to generate a more accurate embedded model output (e.g., a text embedding, a classification embedding, an instruction embedding, an instruction, and/or a classification that is closer to the training output ground truth embedding 2106).

The training set 2102 may be used for training the model 2108. The training set 2102 may include the training input data 2104. In certain embodiments, the training input data 2104 is encoded training input data. The encoded training input data may have been generated by inputting training input data 2104 into an encoder (e.g., an image encoder, a text encoder (e.g., to encode an instruction), an audio encoder). By using encoded training input data (e.g., an encoded image) rather than converting the training data during training time, the network, processing, and energy resources of the training system 2100 can be reduced compared to a system that encodes data at training time. The training set 2102 may be stored in a database of training datasets. The training input data 2104 may be transmitted to the model 2108.

The comparison system 2112 may compare the model output 2110 with the training output ground truth embedding 2106 to determine how similar the training output ground truth embedding 2106 is to the model output 2110. Based on the comparison, the weight adjustment signal 2114 may be transmitted to the model 2108 to cause weights of the model to be adjusted. In certain embodiments, the comparison system 2112 may compare the model output 2110 with the training output ground truth embedding 2106 using a loss function such as a mean squared error (MSE).

In certain embodiments, the model 2108 is trained to generate an image or an instruction (e.g., a decoded image, a decoded text) based on the training input image data (e.g., an embedding). Similar training techniques may be used, but the model output 2110 may represent an image or text and the training output ground truth may represent an image or text, instead of an embedding.

In certain embodiments, the training set 2102 may be used to train the image encoding model. The training input data 2104 may include an image. The training output ground truth embedding 2106 may include a ground truth encoding of the training input data 2104. The model 2108 may include the image encoding model. The model 2108 may receive the training input data 2104 and use the training input data 2104 to generate model output 2110 that includes a generated encoding of the training input data 2104. The comparison system 2112 may compare the training output ground truth embedding 2106 and the model output 2110 to generate a weight adjustment signal 2114 to transmit to the model 2108 that can cause weights of the model 2108 to be adjusted.

In certain embodiments, the training set 2102 may be used to train the text encoding model. The training input data 2104 may include text. The training output ground truth embedding 2106 may include a ground truth encoding of the training input data 2104. The model may include the text encoding model. The model 2108 may receive the training input data and use the training input data 2104 to generate model output 2110 that includes a generated encoding of the training input data 2104. The comparison system 2112 may compare the training output ground truth embedding 2106 and the model output 2110 to generate a weight adjustment signal 2114 to transmit to the model 2108 that can cause weights of the model 2108 to be adjusted. A similar process as described with respect to the image encoder and the text encoder may be performed to train an audio encoder and/or audio decoder.

In certain embodiments, the training set 2102 may be used to train the bed exit classification model. The training input data 2104 may include an encoded input. The training input data 2104 may include an encoding of a first input (e.g., from a first device), an encoding of a second input (e.g., from a second device), and an encoding of any number of other inputs from one or more devices. The training input data 2104 including the encoding of the first input and the encoding of the second input may include a concatenation or vector space summation of the first input and the second input. The training output ground truth embedding may include a classification embedding that maps (e.g., using a data table) to an instruction or an instruction embedding. The model 2108 may include the bed exit classification model (e.g., or another model described herein). The model 2108 may receive the training input data 2104 and use the training input data 2104 to generate model output 2110 that includes a generated encoding of the training input data 2104. The comparison system 2112 may compare the training output ground truth embedding 2106 and the model output 2110 to generate a weight adjustment signal 2114 to transmit to the model 2108 that can cause weights of the model 2108 to be adjusted. The model 2108 can thereby be trained to generate the training output ground truth embedding 21106 (e.g., an instruction) using the training input data 2104.

Other classification models may be trained using the supervised training approach described above. The classification models may be trained to generate an output such that the training output ground truth embedding 2106 is output when the training input data 2104 is input to the model 2108. Training output ground truth embedding 2106 may include an embedding of more than one instructions that may be transmitted to one or more devices. The instruction may indicate the device for the instruction to be transmitted to.

The training output ground truth embedding 2106 may include a ground truth encoding of the training input data 2104. One having ordinary skill in the art with the benefit of the disclosure would recognize other training techniques (e.g., supervised, unsupervised) that may be used to train models described herein. In certain embodiments, an autoencoder training architecture is used to train encoder models and/or decoder models.

Embodiments of a system that uses data obtained and/or generated by devices as input to an instruction generation system to generate one or more instructions enable various technical benefits. Using data generated by medical devices to train an use an AI model can offer significant technical benefits in a healthcare facility. In certain embodiments, by centralizing data processing via use of the instruction generation system (e.g., the AI model), individual devices can offload complex computational tasks, leading to a reduction in processing resources required at the device level. This decreases hardware demands, enabling the use of smaller, less power-intensive devices. Additionally, using an instruction generation system can reduce network usage by filtering and consolidating data before transmission to one or more other devices and thereby reducing the volume of data sent across networks. Reduction in data transmission volume not only lowers bandwidth requirements but also enhances response times for services (e.g., important healthcare related services). The instruction generation system (e.g., including the AI model) can optimize power consumption across systems by predicting and prioritizing device usage, ensuring that only necessary devices remain active. Furthermore, centralized processing by the instruction generation system can enable advanced decision-making without the need for additional materials or duplicate systems, promoting sustainability, reducing hardware requirements, and reducing energy usage.

Additionally, processing data from multiple devices using the instruction generation system can enhance the accuracy and reliability of healthcare related services (e.g., medical actions). The instruction generation system can aggregate and analyze data from various sources, providing a holistic view of a patient's condition that individual devices alone might not achieve. This comprehensive analysis can allow for more precise predictions and recommendations, improving clinical outcomes and resource utilization efficiency. For example, the instruction generation system can combine vital sign readings from multiple monitoring devices to detect subtle patterns or anomalies that might indicate an impending medical emergency, prompting timely intervention by sending an instruction to one or more devices to indicate the pattern, anomaly, and/or an action to be performed. The holistic processing of data as described above can reduce the likelihood of false alarms or missed warnings, enhancing patient safety, care quality, and usage of resources (e.g., energy, memory, processing).

Another benefit is scalability. The instruction generation system can process large volumes of data from numerous devices, making it well-suited for deployment in healthcare facilities like hospitals, where many patients require continuous monitoring. This scalability reduces the need for duplicative computing infrastructure while ensuring that the system can adapt to increased demand without compromising performance. Furthermore, the instruction generation system (e.g., including an AI model) can simplify workflows by triggering specific actions, such as adjusting ventilator settings or alerting healthcare staff, based on data analysis (e.g., and in near real time). This automation can also reduce the burden on medical personnel and reduce the resources used by devices interfaced with by medical personal to perform such tasks.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method implemented by a computer system hosting an artificial intelligence (AI) model, the method comprising:
receiving, from a first device located in a healthcare facility and providing a first type of healthcare related service, a first input related to the first type of healthcare related service;
receiving, from a second device located in the healthcare facility and providing a second type of healthcare related service, a second input related to the second type of healthcare related service;
generating, by at least using the first input and the second input as part of an input to the AI model, an instruction for a third device providing a third type of healthcare related service, the instruction indicated by an output of the AI model, the third type of healthcare related service being based on the first type of healthcare related service and the second type of healthcare related service; and
causing, by at least sending the instruction to the third device, the third device to perform the third type of healthcare related service.

2. The method of claim 1, wherein the first device and the second device are of different types, and wherein the first type of healthcare related service and the second type of healthcare related service are different types of services.

3. The method of claim 1, wherein the first input includes at least one of (i) a medical record or (ii) a message received by the first device from another device located in the healthcare facility.

4. The method of claim 1, wherein the AI model includes a generative AI model, and wherein generating the instruction further comprises inputting a prompt, the first input, and the second input to the generative AI model.

5. The method of claim 1, wherein the AI model includes a classification AI model, wherein the instruction is generated when a first classification is generated, and wherein the method further comprises:
receiving, from a fourth device providing a fourth type of healthcare related service, a third input;
generating, by at least using the first input and the third input as part of the input to the AI model, a second classification and a second instruction for the third device; and
causing, by at least sending the second instruction to the third device the third device to perform the third type of healthcare related service.

6. The method of claim 1, wherein the first device includes a sensor pad or a camera, the third device is an alarm device, and wherein the instruction causes the alarm device to present an alarm indicating patient movement.

7. The method of claim 6, wherein the second device is a medical record storage device that includes a fall risk indication for the patient.

8. The method of claim 1, wherein the third device includes a medical record storage device, and wherein the third type of healthcare related service includes updating a medical record.

9. The method of claim 8, wherein the first device includes at least one of a nurse call device, a user device, a camera, or a sensor pad physically coupled with a bed, and wherein the second device is a different type of device than the first device and includes at least one of a second nurse call device, a second user device, a second camera, or a second sensor pad physically coupled with a second bed.

10. The method of claim 1, wherein the third device includes an environment control device, and wherein the first device includes a medical record storage device.

11. A computer system of a healthcare facility hosting an artificial intelligence (AI) model comprising:
a first device located in the healthcare facility, that provides a first type of healthcare related service;
a second device located in the healthcare facility, that provides a second type of healthcare related service;
a third device that performs a third type of healthcare related service based on an instruction generated by an instruction generation system; and
the instruction generation system comprising:
one or more storage media storing first instructions; and
one or more processors configured to execute the instructions to cause the instruction generation system to:
receive, from the first device, a first input related to the first type of healthcare related service;
receive, from the second device, a second input related to the second type of healthcare related service;
generate, by at least using the first input and the second input as part of an input to the AI model, the instruction for the third device, the instruction indicated by an output of the AI model, the third type of healthcare related service being based on the first type of healthcare related service and the second type of healthcare related service; and
send, the instruction to the third device.

12. The computer system of claim 11, wherein the first device or the second device is a sensor pad, a camera, or a bed.

13. The computer system of claim 11, wherein the third device is a third type of device, is located in the healthcare facility and includes an interface used to perform the third type of healthcare related service.

14. The computer system of claim 11, wherein at least one of the first device, the second device, or the third device include at least one of (i) a hub located in a patient room and configured to be communicatively coupled with a nurse call system of the healthcare facility and another device, (ii) a bed controller, (iii) a television control system, (iv) a medical device, or (v) a sensor pad.

15. The computer system of claim 11, wherein at least one of the first device, the second device, or the third device include a mobile phone, a camera, or a nurse call system controller.

16. The computer system of claim 11, wherein the third device includes at least one of an alarm, a room control device, a messaging device, or a camera.

17. One or more non-transitory computer-readable storage media storing instructions that, upon execution by one or more processors of a system hosting an artificial intelligence (AI) model, cause the system to perform operations comprising:
receiving, from a first device located in a healthcare facility and providing a first type of healthcare related service, a first input related to the first type of healthcare related service;
receiving, from a second device located in the healthcare facility and providing a second type of healthcare related service, a second input related to the second type of healthcare related service;
generating, by at least using the first input and the second input as part of an input to the AI model, an instruction for a third device providing a third type of healthcare related service, the instruction indicated by an output of the AI model, the third type of healthcare related service being based on the first type of healthcare related service and the second type of healthcare related service; and
causing, by at least sending the instruction to the third device the third device to perform the third type of healthcare related service.

18. The non-transitory computer-readable storage media of claim 17, wherein the AI model includes a generative AI model, wherein generating the instruction further comprises inputting a prompt, the first input, and the second input to the generative AI model.

19. The non-transitory computer-readable storage media of claim 17, wherein the AI model includes a classification AI model and the instruction is generated when a first classification is generated and wherein the operations further comprise:
receiving, from a fourth device providing a fourth type of healthcare related service, a third input;
generating, by at least using the first input, and the third input as part of the input to the AI model, a second classification and a second instruction for the third device; and
causing, by at least sending the second instruction to the third device the third device to perform the third type of healthcare related service.

20. The non-transitory computer-readable storage media of claim 17, wherein the first input includes at least one of (i) a medical record or (ii) a message received by the first device, wherein the first device is located in the healthcare facility.
